(19) United States
(12) Patent Application Publication
(10) Pub. No.: US 2010/0324030 A1
(43) Pub. Date: Dec. 23, 2010

(54) 5-Quinoline derivatives having an anti-bacterial activity

(75) Inventors: Glenn Dale, Basel (CH); Sabine Pierau, Freiburg (DE); Michael W. Cappi, Muenchen (DE); Christopher Gray, Zunzgen (CH); Christian Hubschwerlen, Durmenach (FR); Jean P. Surivet, Kembs (FR); Cornelia Zumbrunn, Basel (CH)

Correspondence Address: Edwards Angell Palmer & Dodge LLP, P.O. Box 55874, Boston, MA, 02205, US

(73) Assignee: Morphochem Aktiengesellschaft Fur Kombinatorische Chemie, Munchen (DE)

(21) Appl. No.: 12/082,667

(22) Filed: Apr. 11, 2008

Related U.S. Application Data
(63) Continuation of application No. PCT/EP06/09932, filed on Oct. 13, 2006.

(30) Foreign Application Priority Data
Oct. 13, 2005 (DE) 10 2005 049 039.5
Jun. 22, 2006 (DE) 10 2006 028 649.9

(51) Int. Cl. A61K 31/4709; C07D 413/14; A61K 31/538; C07D 513/04; A61K 31/542; C07D 417/14; A61K 31/5415; C07D 491/056; C07D 405/14; C07D 401/14; A61K 31/498; A61K 31/496; A61P 31/04

(52) U.S. Cl. 514/224.2; 544/105; 514/230.5; 544/48; 544/52; 546/115; 514/302; 546/153; 514/312; 544/353; 514/249; 544/363; 514/253.07

(57) ABSTRACT

The present invention describes novel anti-bacterial compounds of the formula (I).

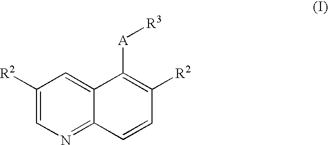

These compounds are, amongst others, of interest as inhibitors of DNA gyrase and topoisomerases, for example of topoisomerase II and IV.

Resistance to the antibiotics currently used has increased appreciably in many countries of the world in recent years and in some cases has assumed alarming proportions. The main problem is that those pathogens exhibit not just a single resistance but, as a rule, multiple resistance. This is true especially of some gram-positive pathogen groups, such as staphylococci, pneumococci and enterococci (S. Ewig et al., Anti-biotika-Resistenz bei Erregem ambulant erworbener Atemwegsinfektionen (Antibiotic resistance in pathogens of outpatient-acquired respiratory tract infections), Chemother. J. 2002, 11, 12-26; F. Tenover, Development and spread of bacterial resistance to antimicrobial agents: an overview, Clin. Infect. Dis. 2001 Sep. 15, 33 Suppl. 3, 108-115).

A long-feared development has recently occurred: In the USA, the first strain of Staphylococcus aureus has been described that is not only resistant to methicillin but also highly resistant to vancomycin (Centers for Disease Control and Prevention, Staphylococcus aureus resistant to vancomycin—United States, 2002, MMWR 2002, 51, 565-567). In addition to hygiene measures in hospitals, therefore, increased efforts are also required to find new antibiotics that as far as possible have a novel structure and a novel mechanism of action so as to be effective against those problem pathogens.

The present invention describes new kinds of compounds having an anti-bacterial activity. These compounds are, amongst others, of interest as inhibitors of DNA gyrase and topoisomerases (for example topoisomerase II and IV).

The present invention relates to compounds of the general formula (I):

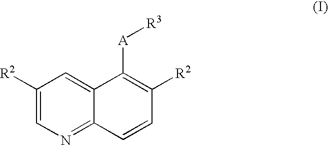

wherein
the rest R¹ is a hydrogen atom, a halogen atom, a hydroxy, an amino, a cyano, a nitro, a thiol, a C₁-C₆ alkyl, a C₂-C₆ alkenyl, a C₂-C₆ alkynyl, a heteroalkyl, an alkyloxy, a heteroalkyloxy, a cycloalkyloxy, an alkylcycloalkyloxy, a heterocycloalkyloxy or a heteroalkylcycloalkyloxy group;
the rest R² is a hydrogen atom, a halogen atom, a hydroxy, an amino, a cyano, a C₁-C₆ alkyl, C₂-C₆ alkenyl, C₂-C₆ alkynyl or a heteroalkyl group;
the rest R³ is a group of the following formula:

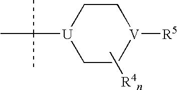

wherein U and V independently of each other are nitrogen atoms or groups of the formula CH or CR⁶;
the rests R⁴ independently of each other are a halogen atom, a hydroxy, an amino, a cyano, a nitro or a thiol group, an alkyl, alkenyl, alkynyl or heteroalkyl group;
n is equal to 0, 1 or 2;
the rest R⁵ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group,
the rests R⁶ independently of each other are a halogen atom, a hydroxy, alkyl, alkenyl, alkynyl or a heteroalkyl group;
A is selected from the following groups: —CR¹⁰(OR¹¹)CR¹²R¹³—, —CR⁸R⁹CR¹⁰(OR¹¹)—, —OCR⁸R⁹CR₁₂R¹³—, CR⁸R⁹CR¹²R¹³O—, —CR⁸R⁹SO₂—, —SO₂CR⁸R⁹—, —CR⁸R⁹NR⁷—, —NR⁷CR⁸R⁹—, —CR⁸R⁹O—, —OCR⁸R⁹—, —CR⁸R⁹S—, —SCR⁸R⁹—, —NR⁷C(═O)—, —C(═O)NR⁷— and —CR⁸R⁹CR¹²R¹³—;
the rest R⁷ is a hydrogen atom, a trifluoromethyl, a C₁-C₆ alkyl, a C₂-C₆ alkenyl, a C₁-C₆ alkoxycarbonyl, a C₁-C₆ alkylcarbonyl or an carbonylamino group, wherein the amino group of the carbonylamino group, if applicable, may be substituted by a C₁-C₆ alkoxycarbonyl, a C₁-C₆ alkylcarbonyl, a C₂-C₆ alkenyloxycarbonyl, a C₂-C₆ alkenylcarbonyl, a C₁-C₆ alkyl, a C₂-C₆ alkenyl and, if applicable, further may be substituted by a C₁-C₆ alkyl or a C₂-C₆ alkenyl group;
the rests R⁸, R⁹, R¹⁰, R¹² and R¹³ independently of each other are a hydrogen atom, a halogen atom, an azide, a trifluoromethyl, hydroxy, amino, C₁-C₆ alkyloxy, C₁-C₆ alkylthio, C₁-C₆ alkyl, C₂-C₆ alkenyl, C₁-C₆ alkoxycarbonyl, C₂-C₆ alkenyloxycarbonyl, C₁-C₆ is alkylsulfonyl, C₂-C₆ alkenylsulfonyl or a sulfonylamino group, wherein the amino group of the sulfonylamino group, if applicable, may be substituted by a C₁-C₆ alkyl or a phenyl group;
the rest R¹¹ is a hydrogen atom, a trifluoromethyl, C₁-C₆ alkyl, C₂-C₆ alkenyl, C₁-C₆ alkoxycarbonyl, C₁-C₆ alkylcarbonyl or an carbonylamino group, wherein the amino group of the carbonylamino group, if applicable, may be substituted by a C₁-C₆ alkoxycarbonyl, C₁-C₆ alkylcarbonyl, C₂-C₆ alkenyloxycarbonyl, C₂-C₆ alkenylcarbonyl, C₁-C₆ alkyl, C₂-C₆ alkenyl and, if applicable, further may be substituted by a C₁-C₆ alkyl or a C₂-C₆ alkenyl group;
or a pharmacologically acceptable salt, solvate, hydrate or a pharmacologically acceptable formulation thereof.

The expression alkyl refers to a saturated, straight-chain or branched hydrocarbon group that contains from 1 to 20 carbon atoms, preferably from 1 to 12 carbon atoms, especially preferably from 1 to 6 carbon atoms, for example a methyl, ethyl, propyl, iso-propyl, n-butyl, iso-butyl, tert-butyl, n-pentyl, n-hexyl, 2,2-dimethylbutyl or n-octyl group.

The expressions alkenyl and alkynyl refer to at least partially unsaturated, straight-chain or branched hydrocarbon groups that contain from 2 to 20 carbon atoms, preferably from 2 to 12 carbon atoms, especially preferably from 2 to 6 carbon atoms, for example an ethenyl, allyl, acetylenyl, propargyl, isoprenyl or hex-2-enyl group. Preferably, alkenyl groups have one or two (especially preferably one) double bond(s), and alkynyl groups have one or two (especially preferably one) triple bond(s).

Furthermore, the terms alkyl, alkenyl and alkynyl refer to groups in which one or more hydrogen atoms have been replaced by a halogen atom (preferably F or CI) such as, for example, a 2,2,2-trichloroethyl or a trifluoromethyl group.

The expression heteroalkyl refers to an alkyl, alkenyl or alkynyl group in which one or more (preferably 1, 2 or 3) carbon atoms have been replaced by an oxygen, nitrogen, phosphorus, boron, selenium, silicon or sulfur atom (preferably by an oxygen, sulfur or nitrogen atom). The expression heteroalkyl furthermore refers to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy.

Examples of heteroalkyl groups are groups of formulae: R^{a}—O—Y^{a}—, R^{a}—N(R^{b})—Y^{a}—, R^{a}—CO—Y^{a}—, R^{a}—O—CO—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d}—N(R^{c})—Y^{a}—, R^{a}—Cs—Y^{a}—, R^{a}—Cs—l (R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a direct bond, a C₁-C₆ alkylene, a C₂-C₆ alkenylene or a C₂-C₆ alkynylene group, wherein each heteroalkyl group contains at least one carbon atom and one or more hydrogen atoms may be replaced by fluorine or chlorine atoms.

Specific examples of heteroalkyl groups are methoxy, trifluoromethoxy, ethoxy, n-propyloxy, isopropyloxy, tert-butyloxy, methoxymethyl, ethoxymethyl, —CH₂CH₂OH, —CH₂OH, methoxyethyl, methylamino, ethylamino, dimethylamino, diethylamino, isopropylethylamino, methylamino methyl, ethylamino methyl, diisopropylamino ethyl, enol ether, dimethylamino methyl, dimethylamino ethyl, acetyl, propionyl, butyryloxy, acetyloxy, methoxycarbonyl, ethoxycarbonyl, N-ethyl-N-methylcarbamoyl or N-methylcarbamoyl. Further examples of heteroalkyl groups are nitrile, isonitrile, cyanate, thiocyanate, isocyanate, isothiocyanate and alkylnitrile groups. An example of a heteroalkylene group is a group of formula —CH₂CH(OH)—.

The expression cycloalkyl refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulfur atom (preferably by an oxygen, sulfur or nitrogen atom). A heterocycloalkyl group has preferably 1 or 2 ring(s) containing from 3 to 10 (especially 3, 4, 5, 6 or 7) ring atoms. The expression heterocycloalkyl refers furthermore to groups in which one or more hydrogen atoms have been replaced by fluorine, chlorine, bromine or iodine atoms or by OH, ═O, SH, ═S, NH₂, ═NH or NO₂ groups. Examples are a piperidyl, piperazinyl, morpholinyl, urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkrylcycloalkyl groups. An alkylcycloalkyl group preferably contains a cycloalkyl group that contains one or two ring systems having from 3 to 10 (especially 3, 4, 5, 6 or 7) ring carbon atoms, and one or two alkyl, alkenyl or alkynyl groups having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulfur atom (preferably by an oxygen, sulfur or nitrogen atom). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl or Ar refers to an aromatic group that contains one or more rings containing from 6 to 14 ring carbon atoms, preferably from 6 to 10 (especially 6) ring carbon atoms. The expression aryl (or Ar, respectively) refers furthermore to groups in which one or more hydrogen atoms have been replaced by fluorine, chlorine, bromine or iodine atoms or by OH, SH, NH₂ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings containing from 5 to 14 ring atoms, preferably from 5 to 10 (especially 5 or 6) ring atoms, and contains one or more (preferably 1, 2, 3 or 4) oxygen, nitrogen, phosphorus or sulfur ring atoms (preferably O, S or N). The expression heteroaryl refers furthermore to groups in which one or more hydrogen atoms have been replaced by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced by an oxygen, nitrogen, silicon, selenium, phosphorus, boron or sulfur atom (preferably oxygen, sulfur or nitrogen), that is to say to groups containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkylheterocycloalkyl, arylalkenyiheterocycloalkyl, arylalkynyiheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl, heteroarylheteroalkylcycloalkyl, heteroarylheteroalkylcycloalkenyl and heteroarylheteroalkylheterocycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

The expressions cycloalkyl, heterocycloalkyl, alkylcycloalkyl, heteroalkylcycloalkyl, aryl, heteroaryl, aralkyl and heteroaralkyl refer to groups in which one or more hydrogen atoms of such groups have been replaced by fluorine, chlorine, bromine or iodine atoms or by OH, ═O, SH, ═S, NH₂, ═NH or NO₂ groups.

The expression “optionally substituted” refers to groups in which one or more hydrogen atoms have been replaced by fluorine, chlorine, bromine or iodine atoms or by OH, ═O, SH, ═S, NH₂, ═NH or NO₂ groups. This expression refers furthermore to groups that are substituted by unsubstituted C₁-C₆ alkyl, C₂-C₆ alkenyl, C₂-C₆ alkynyl, C₁-C₆ heteroalkyl, C₃-C₁₀ cycloalkyl, C₂-C₉ heterocycloalkyl, C₆-C₁₀ aryl, C₁-C₉ heteroaryl, C₇-C₁₂ aralkyl or C₂-C₁₁ heteroaralkyl groups.

Preferred are compounds of the formula (I), wherein A is selected from the following groups: —CH(OH)CH₂—, —CH₂CH(OH)—, —OCH₂CH₂—, —CH₂CH₂O—, —CH₂SO₂—, —SO₂CH₂—, —CH₂N(C₁-C₄ alkyl)-, —N(C₁-C₄ alkyl)CH₂—, —CH₂NH—, —NHCH₂—, —CH₂O—, —OCH₂—, —CH₂S—, —SCH₂—, —N(C₁-C₄ alkyl)C(═O)—, —C(═O)N(C₁-C₄ alkyl)-, —NHC(═O)—, —C(═O)NH— or —CH₂CH₂—.

Especially preferred are compounds of the formula (I), wherein A is a group of the formula —CH(OH)CH₂— or —OCH₂CH₂—.

Again preferably, the rest R¹ is a cyano group, a C₁-C₄ alkyloxy group or a C₁-C₄ heteroalkyloxy group, wherein one or more hydrogen atoms of these groups may be replaced by fluorine atoms.

Especially preferably, the rest R¹ is a methoxy group.

Further preferably, the rest R² is a hydrogen atom or a halogen atom. Especially preferably, the rest R² is a hydrogen, a chlorine or a fluorine atom.

Again preferably, the rest R³ is selected from the following groups:

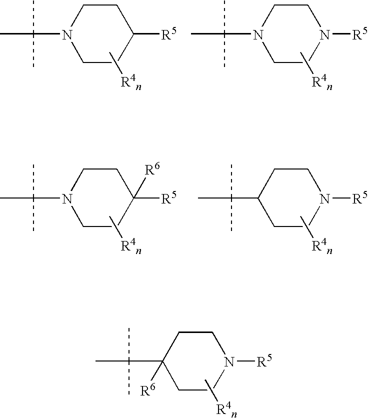

Especially preferably, the rest R³ is selected from the following groups:

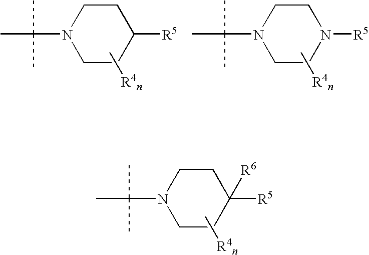

Again preferably, the rests R⁴ are independently of each other a halogen atom, a hydroxy, a cyano, a C₁-C₄ alkyl or a C₁-C₄ heteroalkyl group (for example a hydroxymethyl group).

Especially preferably, the rests R⁴ are independently of each other a fluorine or chlorine atom or a C₁-C₄ heteroalkyl group (for example a hydroxymethyl group).

Preferably, n is equal to 0 or 1; especially preferably, n is equal to 0.

Further preferably, the rest R⁵ is a heteroalkylcycloalkyl or a heteroaralkyl group.

Especially preferably, the rest R⁵ is a group of the formula —B—Y, wherein B is a bond, an alkylene (especially a C₁-C₄ alkylene group), an alkenylene, an alkynylene, a —NH—, —NHSO₂—, —SO₂—, —C(═O), a heteroalkylene (especially a C₁-C₄ heteroalkylene group) or a heterocycloalkylene group, and Y is an aryl, heteroaryl, aralkyl, heteroaralkyl, cycloalkyl, heterocycloalkyl, alkylcycloalkyl, heteroalkylcycloalkyl, heteroarylhetero-cycloalkyl or an arylheterocycloalkyl group (especially a heterocycloalkyl, a heteroaryl, aralkyl, heteroaralkyl, a heteroarylheterocycloalkyl or an arylheterocycloalkyl group).

Preferably, B is a bond or a group of the formula —NH—, —NHCH₂—, —CH₂NH—, —NHCH₂CH₂—, —CH₂CH₂NH—, —NHCH₂CH₂CH₂—, —CH₂CH₂CH₂NH—, —CH₂—, —CH₂CH₂—, —CH₂CH₂CH₂—, —NHC(═O)—, —C(═O)NH—, —CH(OH)—, —CH₂CH(OH)—, —CH(OH)CH₂—, —NHSO₂—, —SO₂NH—, —SO₂—, —C(═S)NH—, —NHC(═S)—, —C(═NOH)—, —CH₂C(═NOH)—, —C(═NOH)CH₂—, —C(═O)—, —C(═O)—C(═O)—, —CH₂C(═O)—, —C(═O)CH₂—, —N(C₁-C₄ alkyl)CH₂—, —CH₂N(C₁-C₄ alkyl)- or a piperazine group.

Especially preferably, B is a bond or a group of the formula —NHCH₂—, —CH₂NH—, —CH₂—, —CH₂CH₂—, —CH₂CH₂CH₂—, —NHC(═O)—, —C(═O)NH—, —CH₂CH(OH)—, —CH(OH)CH₂—, —NHSO₂—, —SO₂NH—, —SO₂—, —C(═O)— or a piperazine group.

Preferably, Y is a bicyclic system, wherein the two rings independently of each other are a cycloalkyl, a heterocycloalkyl, an aryl (especially a phenyl ring) or a heteroaryl ring, and each have from 3 to 8 ring atoms (preferably 5, 6 or 7 ring atoms) (especially preferably, the heteroaryl ring has 5 or 6 ring atoms), and if applicable, the system may be substituted (for example by F, ═O, methyl, trifluoromethyl, methoxy, —C(═O)OH, cyclopropyl}

Again preferably, Y is a group of the formula —Y¹—Y², wherein Y¹ is a bond, an alkylene (especially a C₁-C₄ alkylene group), an alkenylene (especially a C₂-C₄ alkenylene group), an alkynylene, a —NH—, a —S—, a —O—, a —NHC(═O)—, a —C(═O)NH— or a heteroalkylene group (especially a C₁-C₄ heteroalkylene group), and Y² is an aryl, heteroaryl, aralkyl, heteroaralkyl, cycloalkyl, heterocycloalkyl, alkylcycloalkyl, heteroalkylcycloalkyl, heteroarylheterocycloalkyl or an arylheterocycloalkyl group (especially a heterocycloalkyl, an aryl, a heteroaryl, aralkyl, heteroaralkyl, a heteroarylheterocycloalkyl or an arylheterocycloalkyl group). Especially preferably, Y¹ is a bond or a group of the formula —CH═CH—, —CH₂CH₂—, —S—, —CH₂O—, —C(═O)NH—, —NH— or —CH₂C(═O)—, and Y² is an optionally substituted phenyl group or heteroaryl group having 5 or 6 ring atoms.

Especially preferably, Y has one of the following structures:

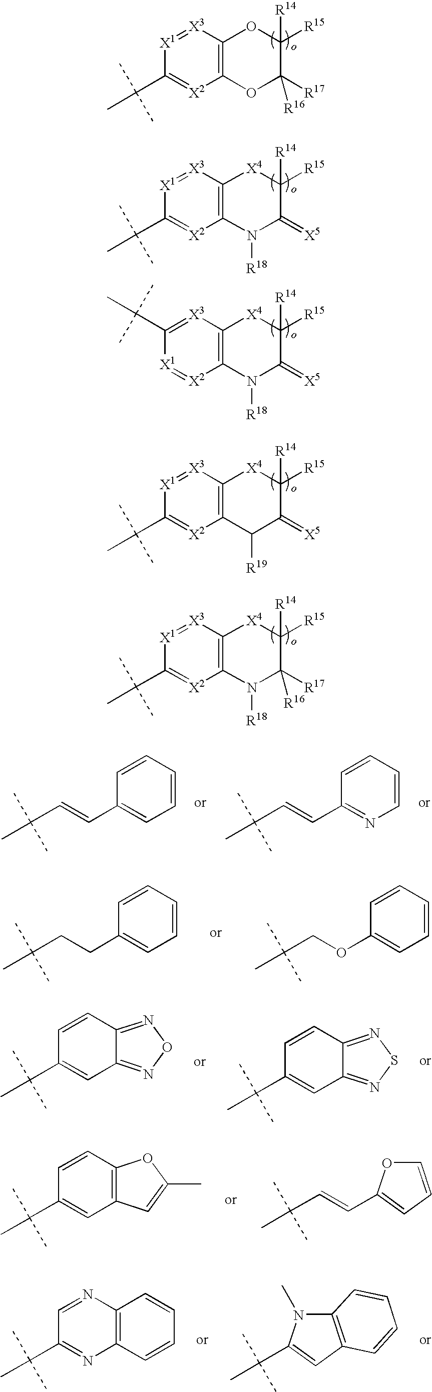

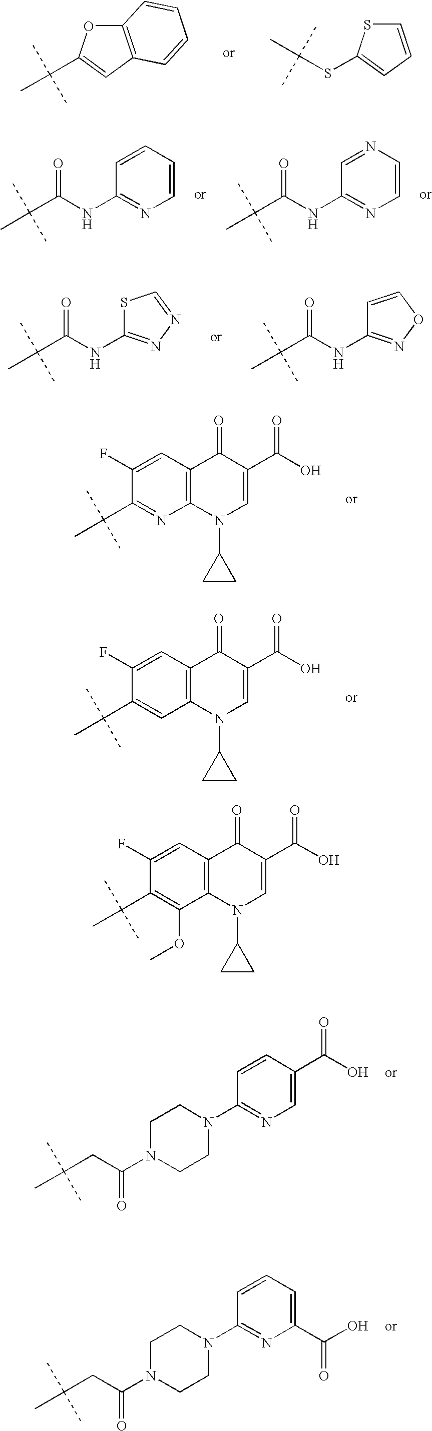

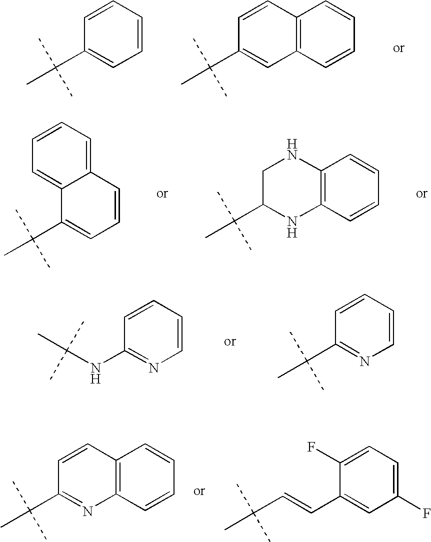

wherein X¹, X² and X³ independently of each other are nitrogen atoms or groups of the formula CR²⁰, X⁴ and X⁵ independently of each other are oxygen or sulfur atoms or groups of the formula NR²¹, o is equal to 0, 1 or 2, R¹⁴, R¹⁵, R¹⁶, R¹⁷, R¹⁹ and R²⁰ independently of each other are hydrogen atoms, halogen atoms, hydroxy, C₁-C₆ alkyl, C₂-C₆ alkenyl, C₂-C₆ alkynyl or C₁-C₆ heteroalkyl groups, and R¹⁸ and R²¹ independently of each other are hydrogen atoms, C₁-C₆ alkyl, C₂-C₆ alkenyl, C₂-C₆ alkynyl or C₁-C₆ heteroalkyl groups.

Especially preferably, Y has one of the following structures:

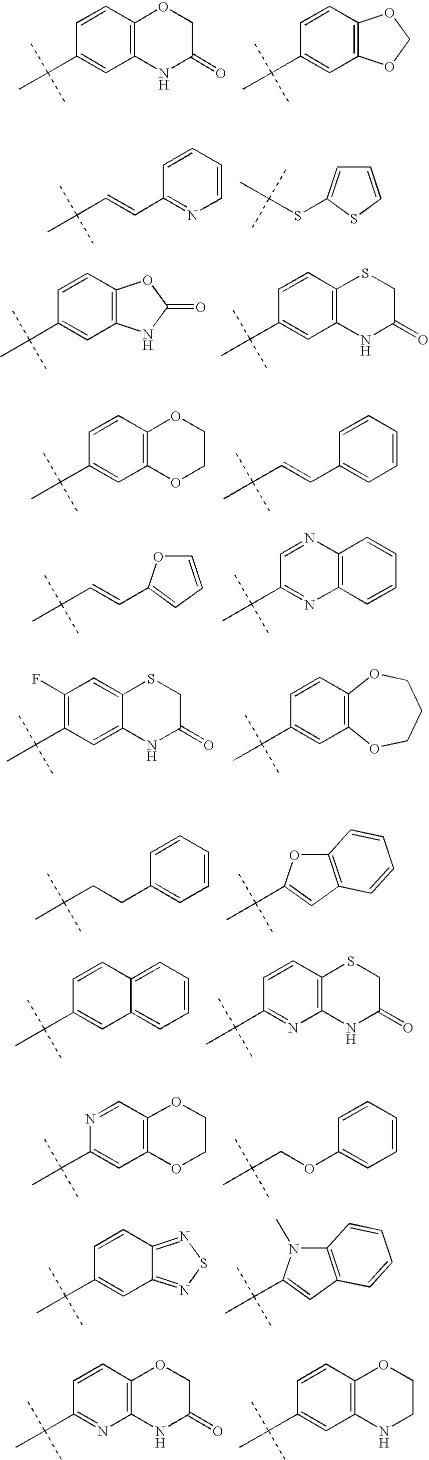

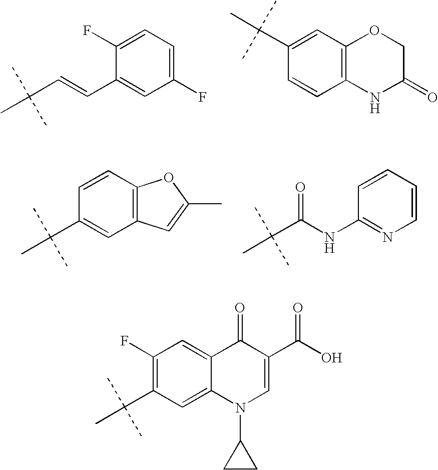

Especially preferably, Y has one of the following structures:

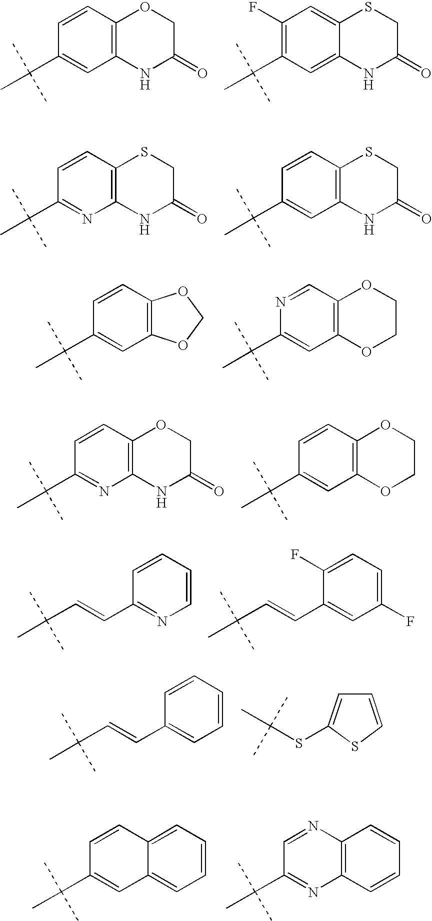

Further preferably, the rests R⁶ independently of each other are a halogen atom, a hydroxy, a C₁-C₄ alkyl or a C₁-C₄ heteroalkyl group (for example a hydroxyethyl group).

Again further preferably, the rests R⁶ independently of each other are a fluorine or a chlorine atom or a hydroxy, a C₁-C₄ alkyloxy, a C₁-C₄ heteroalkyl (for example a hydroxyethyl group) or a C₃-C₆ dialkylamino methyl group, wherein one or more hydrogen atoms of these groups may be replaced by fluorine atoms.

Especially preferably, the rests R⁶ independently of each other are a C₁-C₄ heteroalkyl group (for example a hydroxyethyl group).

Especially preferred are compounds of the formula (II):

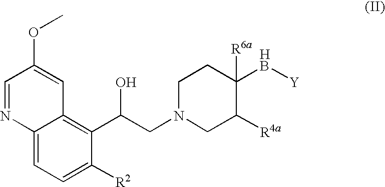

wherein the rests R², R^{4a} and R^{6a} independently of each other are a hydrogen atom or a halogen atom or a C₁-C₄ heteroalkyl group (for example a hydroxymethyl or hydroxy-ethyl group) (especially R² is a hydrogen atom, and R^{4a} and R^{6a} are a hydrogen or fluorine atom or a C₁-C₄ heteroalkyl group). B and Y are as defined above. Especially, B is a bond or a group of the formula —NHCH₂—, —NHC(═O)— or —NHSO₂—.

Especially preferred are compounds of the formula (III):

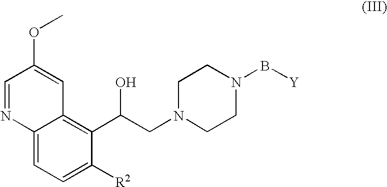

wherein the rest R² is a hydrogen atom or a halogen atom (especially R² is a hydrogen atom). B and Y are as defined above. Especially, B is a bond or a group of the formula —CH₂CH₂—, —CH₂CH₂CH₂— or —CH₂CH(OH)—.

Especially preferred are compounds of the formula (IV):

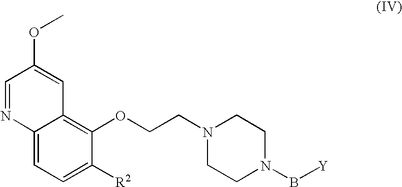

wherein the rest R² is a hydrogen atom or a halogen atom (especially R² is a hydrogen atom). B and Y are as defined above. Especially, B is a bond or a group of the formula —CH₂—, —CH₂CH₂—, —SO₂— or —C(═O)—.

It is especially preferred to combine the preferred embodiments for each generic group in the formulae (I), (II), (III) and (IV) in any possible manner.

Due to their substitution, the compounds of the formulae (I) to (IV) may contain one or more chirality centers. Thus, the present invention comprises both all pure enantiomers and all pure diastereomers, and also the mixtures thereof in any mixing ratio.

Furthermore, the present invention comprises also all cis/trans isomers of the compounds of the general formulae (I) to (IV), as well as mixtures thereof. Additionally, the present invention comprises all tautomeric forms of the compounds according to the formulae (I) to (IV).

The therapeutic use of compounds according to the formulae (I) to (IV), their pharmacologically acceptable salts, solvates and hydrates, respectively, as well as formulations and pharmaceutical compositions also lie within the scope of the present invention.

The pharmaceutical compositions according to the present invention comprise at least one compound of the formulae (I) to (IV) as an active ingredient and, optionally, carrier substances and/or adjuvants.

Examples of pharmacologically acceptable salts of the compounds of the formulae (I) to (IV) are salts of physiologically acceptable mineral acids, such as hydrochloric acid, sulfuric acid and phosphoric acid, or salts of organic acids, such as methanesulfonic acid, p-toluenesulfonic acid, lactic acid, acetic acid, trifluoroacetic acid, citric acid, succinic acid, fumaric acid, maleic acid and salicylic acid. Further examples of pharmacologically acceptable salts of the compounds of the formulae (I) to (IV) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts. Compounds of the formulae (I) to (IV) may be solvated, especially hydrated. The hydration may take place, for example, during the preparation process or as a consequence of the hygroscopic nature of the initially anhydrous compounds of the formulae (I) to (IV). When the compounds of the formulae (I) to (IV) comprise asymmetric C-atoms, they may be present either in the form of achiral compounds, diastereoisomeric mixtures, mixtures of enantiomers or in the form of optically pure compounds.

The pro-drugs (for example R. B. Silverman, Medizinische Chemie, VCH Weinheim, 1995, chapter 8, p. 361 ff) to which the present invention also relates consist of a compound of the formulae (I) to (IV) and at least one pharmacologically acceptable protecting group which will be removed under physiological conditions, such as, for example, an alkoxy, aralkyloxy, acyl or acyloxy group, such as, for example a methoxy, ethoxy, benzyloxy, acetyl or acetyloxy group.

The present invention relates also to the use of those active ingredients in the preparation of medicaments. In general, compounds of the formulae (I) to (IV) are administered either individually, or in combination with any other desired therapeutic agent, using the known and acceptable methods. Such therapeutically useful agents may be administered, for example, by one of the following routes: orally, for example in the form of dragées, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or a spray; transdermally or intranasally. For the preparation of such tablets, pills, semi-solid substances, coated tablets, dragées and hard gelatine capsules, the therapeutically usable product may be mixed with pharmacologically inert, inorganic or organic pharmaceutical carrier substances, for example with lactose, sucrose, glucose, gelatine, malt, silica gel, starch or derivatives thereof, talcum, stearic acid or salts thereof, skimmed milk powder, and the like. For the preparation of soft capsules, pharmaceutical carrier substances such as, for example, vegetable oils, petroleum, animal or synthetic oils, wax, fat and polyols may be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils may be used. For suppositories, pharmaceutical carrier substances such as, for example, vegetable oils, petroleum, animal or synthetic oils, wax, fat and polyols may be used. For aerosol formulations, compressed gases that are suitable for this purpose, such as, for example, oxygen, nitrogen and carbon dioxide may be used. The pharmaceutically acceptable agents may also comprise additives for preserving and stabilising, emulsifiers, sweeteners, flavourings, salts for altering the osmotic pressure, buffers, encapsulation additives and antioxidants.

The compounds of the formulae (I), (II), (III) and (IV) have improved properties when compared to antibacterial compounds known in the state of the art. Especially, an improved antibacterial activity, an improved solubility and improved PK properties have to be mentioned in this context.

Combinations with other therapeutic agents may comprise other antimicrobial and anti-fungal active ingredients.

For the prevention and/or treatment of the diseases described above, the dose of the biologically active compound according to the invention may vary within wide limits and may be adjusted to individual requirements. Generally, a dose of from 10 mg to 4000 mg per day is suitable, a preferred dose being from 50 to 3000 mg per day. In suitable cases, the dose may also be below or above the stated values. The daily dose may be administered as a single dose or in a plurality of doses. A typical individual dose contains approximately 50 mg, 100 mg, 250 mg, 500 mg, 1 g or 2 g of the active ingredient.

### EXAMPLES

### Example 1

### 6-({1-[2-Hydroxy-2-(3-methoxy-quinolin-5-yl)-ethyl]-piperidin-4-ylamino}methyl)-4H-benzo[1,4]oxazin-3-one (enantiomer 2)

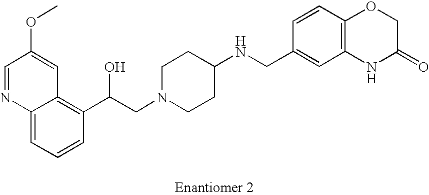

### 1a) 3,5-Dibromo-quinoline

3-Bromoquinoline (250 g) was added in drops to ice cold concentrated sulfuric acid (625 ml), so that the temperature did not rise above 15° C. Thereafter, N-bromo-succinimide (240 g) was added slowly and in portions, so that the temperature did not rise above 20° C., and the reaction mixture was stirred at room temperature overnight. The reaction mixture was poured on ice (10 kg) and during cooling, mixed with solid sodium hydroxide. The suspension being produced was filtered, the solid was washed with water and dried at 40° C. under vacuum. The solid was resuspended in methanol (1.5 l) and then heated to reflux. After cooling, the solid was filtered, rinsed again with cold methanol (500 ml), and the filtrate was concentrated. The crude product was purified by flash chromatography (silica gel, acetic acid ethyl ester:heptane: 1:29, 1:19, 1:9) and resulted in the desired product (151 g).

¹H NMR (300 MHz, CDCl₃): δ: 8.85 (d, 1H), 8.65-8.64 (m, 1H), 7.99 (d, 1H), 7.78 (d, 1H), 7.56-7.49 (m, 1H)

### 1b) 5-Bromo-3-methoxy-quinoline

3,5-Dibromoquinoline (1a) (150 g) was added to a stirred suspension of sodium methylate (35.78 g) in dry DMPU (1.5 l) at 100° C. and then heated to 125° C. for 90 minutes. After cooling, the reaction mixture was poured on ice (7.5 kg) and stirred overnight. The produced solid was separated by filtration, washed with water and dried at 40° C. under vacuum. The crude product was purified by flash chromatography (silica gel, acetic acid ethyl ester:heptane: 1:19, 1:4) and resulted in the desired product (65.2 g).

¹H NMR (300 MHz, CDCl₃): δ: 8.60 (d, 1H), 7.95 (d, 1H), 7.72 (d, 1H), 7.65 (d, 1H), 7.37-7.31 (m, 1H), 3.93 (s, 3H)

### 1c) 3-Methoxy-5-vinyl-quinoline

Under a nitrogen gas atmosphere, tetrakis(triphenylphosphine) palladium(0) (1.155 g) was added to a solution of methoxy quinoline (1b) (9.52 g) in dry 1,2-dimethoxyethane (450 ml) at room temperature and stirred for 20 minutes. Thereafter, potasslum carbonate (5.57 g), water (120 ml) and 2,4,6-trivinylcycloboroxane pyridine complex (3.85 g —O'Sheas'reagent—see J. Org. Chem., Vol. 67 (2002), 4968-4971) were added and heated to 100° C. for 4 hours. After cooling to room temperature, water (200 ml) was added, and the reaction mixture was extracted with acetic acid ethyl ester (4×150 ml). The combined organic phases were dried over sodium sulfate, filtered and concentrated. The crude product was purified by flash chromatography (silica gel, heptane:acetic acid ethyl ester: 9:1, 3:2) and resulted in the desired product (7.41 g).

¹H NMR (300 MHz, CDCl₃): δ: 8.60 (d, 1H), 7.91 (d, 1H), 7.57-7.41 (m, 3H), 7.28-7.22 (m, 1H), 5.72 (dd, 1H), 5.43 (dd, 1H), 3.87 (s, 3H)

### 1d) 1-(3-Methoxy-quinolin-5-yl)-ethan-1,2-diol (enantiomer 2)

AD mix alpha (90.2 g) and methane sulfonic acid amide (7.6 g) were dissolved in water (280 ml) and tert-butanol (280 ml) at room temperature. The orange solution was cooled to 0° C., vinyl quinoline (1c) (14.4 g) was added and stirred at 0-4° C. for 2 days. Thereafter, sodium pyrosulfite (108 g) was added at 0° C. and stirred for 30 minutes at this temperature. After heating to room temperature, the reaction mixture was extracted with acetic acid ethyl ester (5×150 ml). The combined organic phases were dried over sodium sulfate, filtered and concentrated. The crude product was purified by flash chromatography (silica gel, dichloromethane:methanol: 29:1, 4:1) and resulted in the desired product (14.91 g).

¹H NMR (300 MHz, d₆-DMSO): δ: 8.65 (d, 1H), 7.88-7.85 (m, 2H), 7.66 (d, 1H), 7.58-7.53 (m, 1H), 5.51 (d, 1H), 5.31-5.26 (m, 1H), 4.87-4.84 (m, 1H), 3.96 (s, 3H), 3.67-3.57 (m, 2H)

### 1e) Toluene-4-sulfonic acid 2-hydroxy-2-(3-methoxy-quinolin-5-yl)-ethyl ester (enantiomer 2)

Under a nitrogen gas atmosphere, dibutyl tin oxide (0.33 g), p-toluene sulfonic acid (12.78 g) and triethylamine (9.33 ml) were added to a suspension of the diol (1d) (14.4 g) in dry dichloromethan (150 ml) at room temperature under stirring. The reaction mixture was stirred for 14 hours, and then quenched with water (150 ml), and the organic phase was separated. The aqueous phase was extracted again twice with dichloromethane (150 ml each). The combined organic phases were washed with water (150 ml) and a saturated solution of sodium chloride (150 ml), dried over sodium sulfate, filtered and concentrated. The crude product was purified by flash chromatography (silica gel, dichloromethane:methanol: 9:1) and resulted in the desired product (16.12 g).

¹H NMR (300 MHz, d₆-DMSO): δ: 8.63 (d, 1H), 7.89 (d, 1H), 7.67-7.62 (m, 2H), 7.58-7.47 (m, 3H), 7.27 (d, 2H), 6.05 (bs, 1H), 5.56 (bs, 1H), 4.25 (dd, 1H), 4.14 (dd, 1H), 3.89 (s, 3H), 2.34 (s, 3H)

### 1f) 3-Methoxy-5-oxiranyl-quinoline (enantiomer 2)

A solution of the tosylate (1e) (15.97 g) in diethyl ether (300 ml) was mixed under stirring with a 2 N solution of sodium hydroxide (110 ml) at room temperature. The two-phase mixture was stirred at room temperature for 3 hours, and then the organic phase was separated. The aqueous phase was extracted again three times with diethyl ether (150 ml). The combined organic phases were dried over sodium sulfate, filtered and concentrated. The crude product was purified by flash chromatography (silica gel, acetic acid ethyl ester:heptane: 1:1) and resulted in the desired product (5.8 g)

¹H NMR (300 MHz, CDCl₃): δ: 8.64 (d, 1H), 7.94 (dd, 1H), 7.59 (d, 1H), 7.48-7.39 (m, 2H), 4.30 (m, 1H), 3.91 (s, 3H), 3.22 (dd, 1H), 2.81 (dd, 1H)

### 1g) {1-[2-Hydroxy-2-(3-methoxy-quinolin-5-yl)-ethyl]-piperidin-4-yl}carbamic acid tert-butyl ester (enantiomer 2)

The epoxide (1f) (689 mg) and 4-(tert-butoxycarbonylamino) piperidine (686 mg) were dissolved in DMF (11 ml), mixed with potassium carbonate (497 mg) and lithium perchlorate (364 mg), and stirred at 80° C. for 2 days. The solution was concentrated, the residue was dissolved in dichloromethane and extracted with water and a saturated solution of sodium chloride. The organic phase was dried over magnesium sulfate, filtered and concentrated. The residue was purified by flash chromatography (silica gel, dichloromethane:methanol: 97:3) and resulted in the desired product (1.22 g).

¹H NMR (300 MHz, CDCl₃): δ: 8.56 (d, 1H), 7.92 (m, 1H), 7.64 (m, 1H), 7.48-7.43 (m, 1H), 5.72 (bs, 1H), 4.51 (m, 1H), 3.85 (s, 3H), 3.60-3.46 (m, 1H), 3.29-3.26 (m, 2H), 2.84-2.73 (m, 2H), 2.67-2.40 (m, 2H), 2.09-1.95 (m, 2H), 1.84-1.64 (m, 2H), 1.38 (s, 9H)

### 1h) 2-(4-Amino-piperidin-1-yl)-1-(3-methoxy-quinolin-5-yl)-ethanol (enantiomer 2)

The Boc-amine (1g) (1.22 g) was dissolved in dichloromethane (23 ml), and treated with trifluoroacetic acid (2.3 ml) at 0-5° C. and stirred overnight at room temperature. The solution was adjusted to an alkaline pH value with a 2 N solution of sodium hydroxide, and the phases were separated. The aqueous phase was extracted with dichloromethane. The combined organic phases were washed with a saturated solution of sodium chloride, dried over magnesium sulfate, filtered and concentrated. The residue was purified by flash chromatography (silica gel, dichloromethane:methanol: 9:1+1% ammonia) and resulted in the desired product (557 mg).

¹H NMR (300 MHz, d₆-DMSO): δ: 8.66-8.63 (m, 1H), 7.87-7.82 (m, 2H), 7.67 (m, 1H), 7.58-7.51 (m, 1H), 5.43-5.39 (m, 1H), 5.26-5.23 (m, 1H), 3.95 (s, 3H), 2.99-2.85 (m, 2H), 2.62-2.55 (m, 1H), 2.19-1.99 (m, 2H), 1.72-1.57 (m, 2H)

### 1i) (4-Formyl-2-nitro-phenoxy)acetic acid ethyl ester

4-Hydroxy-3-nitro benzaldehyde (25 g) was dissolved in DMF (250 ml). Thereafter, potassium carbonate (22.7 g) was added and chloroacetic acid ethyl ester (23.2 ml) was added in drops. The solution was stirred at 50° C. for 2 days and at room temperature for further 2 days, then diluted with water and extracted with acetic acid ethyl ester. The combined organic phases were washed with water, dried over magnesium sulfate, filtered and concentrated, and resulted in the desired product (37.8 g).

¹H NMR (300 MHz, d₆-DMSO): δ: 9.96 (s, 1H), 8.44 (s, 1H), 8.15 (dd, 1H), 7.52 (d, 1H), 5.17 (s, 2H), 4.18 (q, 2H), 1.21 (t, 3H)

### 1j) 3-Oxo-3,4-dihydro-2H-benzo[1,4]oxazine-6-carbaldehyde

Phenoxyacetic acid ethyl ester (1i) (37.7 g) was dissolved in acetic acid (1000 ml). Thereafter, iron powder (83 g) was added and stirred at 80° C. for 1.5 hours. The reaction mixture was filtered through Decalit and concentrated. The residue was resuspended or redissolved, respectively, in a saturated solution of sodium hydrogencarbonate and extracted with acetic acid ethyl ester. The combined organic phases were dried over magnesium sulfate, filtered and concentrated. The residue was mixed with ether, the precipitate was filtered and resulted in the desired product (20 g).

¹H NMR (300 MHz, d₆-DMSO): δ: 11.00 (bs, 1H), 9.84 (s, 1H), 7.54 (dd, 1H), 7.39 (d, 1H), 7.14 (d, 1H), 4.72 (s, 2H)

### 1k) Title Compound

The amine (1h) (100 mg) was dissolved in 1,2-dichloroethane (6 ml) and methanol (2 ml), and mixed with a molecular sieve 3A (1.00 g) and the aldehyde (1j) (71 mg). The mixture was stirred overnight at room temperature. Then, sodium borohydride (13 mg) was added thereto, and the mixture was stirred for 4 hours at room temperature. The molecular sieve was separated by filtration, and the filtrate was washed with a saturated solution of sodium hydrogencarbonate and a saturated solution of sodium chloride. The organic phase was dried over magnesium sulfate, filtered and concentrated. The residue was purified by flash chromatography (silica gel, dichloromethane:methanol: 9:1+1% ammonia) and resulted in the desired product (70 mg).

¹H NMR (300 MHz, d₆-DMSO): δ: 10.78 (s, 1H), 8.66 (d, 1H), 7.96 (s, 1H), 7.88-7.84 (m, 2H), 7.70-7.68 (m, 1H), 7.59-7.54 (m, 1H), 7.00-6.91 (m, 3H), 5.75 (s, 1H), 5.49-5.46 (m, 1H), 4.56 (s, 2H), 3.96 (s, 3H), 2.82 (s, 2H), 3.12-3.03 (m, 2H), 2.72-2.60 (m, 2H), 2.30-2.10 (m, 2H), 2.00-1.85 (m, 2H), 1.58-1.36 (m, 3H)

### Example 2

### 6-({1-[2-Hydroxy-2-(3-methoxy-quinolin-5-yl)-ethyl]-piperidin-4-ylamino}-methyl)-4H-pyrido[3,2-b][1,4]thiazin-3-one (enantiomer 2)

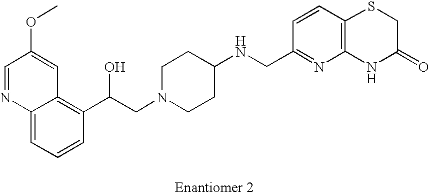

### 2a) N-(6-Methyl-pyridin-2-yl)-acetamide

A solution of 3-amino-6-picoline (39 g) in acetic acid anhydride (200 ml) was heated to 70° C. for 90 minutes and subsequently concentrated. The residue was resuspended or redissolved, respectively, in water (500 ml), adjusted to a pH value of 8 with solid sodium hydrogencarbonate and extracted with acetic acid ethyl ester (2×200 ml). The combined organic phases were washed with a saturated solution of sodium chloride, dried over magnesium sulfate, filtered and concentrated, and resulted in the desired product (53.3 g).

¹H NMR (300 MHz, CDCl₃): δ: 8.43 (bs, 1H), 8.00 (d, 1H), 7.62-7.57 (m, 1H), 6.89 (d, 1H), 2.45 (s, 3H), 2.18 (s, 3H)

### 2b) 6-Acetylamino-pyridine-2-carboxylic acid

A suspension of the acetamide (2a) (53.3 g) in water (530 ml) was heated to 75° C., until a homogenous solution had formed. Potassium permanganate (133 g) was added in small portions within 1.25 hours (the reaction temperature in the reaction flask was controlled carefully). After stirring for 3 hours at 75° C., the reaction solution was filtered through Celit in hot state and rinsed again with hot water. The filtrate was concentrated to about 100 ml, and concentrated hydrochloric acid was added, until a white precipitate had formed. The white solid was separated by filtration, dried and resulted in the desired product (32 g).

¹H NMR (300 MHz, d₆-DMSO): δ: 10.85 (s, 1H), 8.26 (d, 1H), 7.97-7.72 (m, 1H), 7.73 (dd, 1H), 2.11 (s, 3H)

### 2c) 6-Amino-pyridine-2-carboxylic acid methyl ester

The acid (2b) (18 g) was suspended in methanol, saturated with HCl gas and heated overnight under reflux. After cooling, the reaction mixture was concentrated, and the residue was resuspended or redissolved, respectively, in water and dichloromethane. Solid sodium hydrogencarbonate was added and the phases were separated. The aqueous phase was extracted with dichloromethane. The combined organic phases were washed with a saturated solution of sodium chloride, dried over magnesium sulfate, filtered and concentrated. The residue was purified by flash chromatography (silica gel, dichloromethane:acetic acid ethyl ester: 1:1) and resulted in the desired product (9.64 g).

¹H NMR (300 MHz, CDCl₃): δ: 7.52-7.41 (m, 2H), 6.66 (dd, 1H), 5.12 (bs, 2H), 3.91 (s, 3H)

### 2d) 6-Amino-5-bromo-pyridine-2-carboxylic acid methyl ester

To a solution of the ester (2c) (9.64 g) in chloroform (408 ml), a solution of bromine (3.35 ml) in chloroform (70 ml) was added in drops within 60 minutes. After the reaction mixture had been stirred at room temperature for 40 hours, a saturated solution of sodium thiosulfate (150 ml) was added, and the phases were separated. The aqueous phase was extracted with dichloromethane. The combined organic phases were washed with a saturated solution of sodium chloride, dried over magnesium sulfate, filtered and concentrated. The residue was purified by flash chromatography (silica gel, hexane:acetic acid ethyl ester: 2:1) and resulted in the desired product (1.8 g).

¹H NMR (300 MHz, CDCl₃): δ: 7.73 (d, 1H), 7.29 (d, 1H), 5.39 (bs, 2H), 3.90 (s, 3H)

### 2e) 3-Oxo-3,4-dihydro-2H-pyrido[3,2-b][1,4]thiazine-6-carboxylic acid methyl ester

To a solution of methyl thioglycolate (2.4 ml) in DMF (75 ml), sodium hydride (1.1 g) was added. After one hour, the bromopyridine (2d) (5 g) was added and stirred for 12 hours at room temperature. The reaction solution was diluted with water (150 ml).

The solid was separated by filtration, washed with a small amount of acetic acid ethyl ester and acetonitrile, and resulted in the desired product (1.65 g).

¹H NMR (300 MHz, d₆-DMSO): δ: 11.29 (s, 1H), 7.97 (d, 1H), 7.66 (d, 1H), 3.86 (s, 3H), 3.64 (s, 2H), 3.33 (s, 3H)

### 2f) 3-Oxo-3,4-dihydro-2H-pyrido[3,2-b][1,4]thiazine-6-carboxylic acid

To a solution of the ester (2e) (2.33 g) in dioxane (354 ml) and water (90 ml), a 2 N solution of sodium hydroxide (24 ml) was added in drops within 2 hours and then stirred at room temperature overnight. The solution was concentrated and the pH value was adjusted to 4 with a 2 N solution of hydrochloric acid. The produced solid was separated by filtration, washed with a small amount of water and dried overnight under vacuum, and resulted in the desired product (1.72 g).

MS (EI): m/z: 211 [M+H]⁺

### 2g) 6-Hydroxymethyl-4H-pyrido[3,2-b][1,4]thiazin-3-one

To a solution of the acid (2f) (1.72 g) in THF (82 ml), triethylamine (1.4 ml) and isobutyl chloroformate (1.2 ml) were added at −10° C. After 25 minutes, the solution was filtered through Celit into an ice cold solution of sodium borohydride (1.1 g) in water (28 ml), stirred at this temperature for 30 minutes, and the pH value was adjusted to 7 with a 0.2 N solution of hydrochloric acid. After the concentration step, the produced solid was separated by filtration, washed with water and dried, and resulted in the desired product (1.1 g).

MS (EI): m/z: 197 [M+H]⁺

### 2h) 3-Oxo-3,4-dihydro-2H-pyrido[3,2-b][1,4]thiazine-6-carbaldehyde

To a solution of the alcohol (2g) (1.1 g) in dichloromethane (100 ml) and THF (100 ml), manganese dioxide (2.5 g) was added. After stirring at room temperature for 90 minutes, additional manganese dioxide (3g) was added, stirred for further 2 hours at room temperature, and then the reaction mixture was filtered through Celit. The filtrate was concentrated and resulted in the desired product (598 mg).

¹H NMR (300 MHz, CDCl₃): δ: 9.85 (s, 1H), 8.40 (bs, 1H), 7.74 (d, 1H), 7.55 (d, 1H), 3.50 (s, 2H)

### 2i) Title Compound

This compound was prepared as in example 1k starting from the aldehyde (2h) in a yield of 96%.

¹H NMR (300 MHz, d₆-DMSO): δ: 10.85 (s, 1H), 8.65 (d, 1H), 7.87-7.82 (m, 2H), 7.74-7.67 (m, 2H), 7.58-7.51 (m, 1H), 7.11-7.05 (m, 1H), 5.50-5.36 (m, 1H), 5.30-5.19 (m, 1H), 3.95 (m, 1H), 3.72 (s, 2H), 3.53 (s, 2H), 3.02-2.87 (m, 2H), 2.65-2.54 (m, 2H), 2.44-2.32 (m, 1H), 2.21-2.02 (m, 2H), 1.86-1.70 (m, 3H), 1.42-1.13 (m, 3H)

### Example 3

### 7-Fluoro-6-({1-[2-hydroxy-2-(3-methoxy-quinolin-5-yl)-ethyl]-piperidin-4-ylamino}-methyl)-4H-benzo[1,4]thiazin-3-one (enantiomer 2)

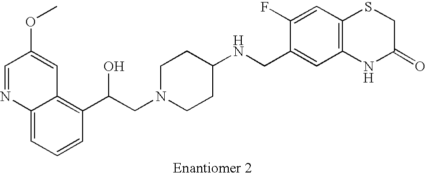

### 3a) 2,4-Difluorobenzoic acid ethyl ester

2,4-Difluorobenzoic acid (5.00 g) was dissolved in ethanol (50 ml), and HCl gas was passed through the solution for 20 minutes. Thereafter, the solution was heated under reflux for 5 hours, the solution was concentrated and the residue was dissolved in ether. The organic phase was washed with a 1 N solution of sodium hydroxide and a saturated solution of sodium chloride, dried over magnesium sulfate, filtered and concentrated, and resulted in the desired product (3.8 g).

¹H NMR (300 MHz, CDCl₃): δ: 8.05-7.95 (m, 1H), 6.99-6.82 (m, 2H), 4.40 (q, 2H), 1.22 (t, 3H)

### 3b) 2,4-Difluoro-5-nitro-benzoic acid ethyl ester

The ethyl ester (3a) (3.8 g) was dissolved in fuming nitric acid (3 ml) and concentrated sulfuric acid (3 ml) at 0° C. and stirred for 2.5 hours. Thereafter, the reaction mixture was diluted with water (10 ml) and extracted with dichloromethane (200 ml). The organic phase was washed with a saturated solution of sodium chloride, dried over magnesium sulfate, filtered and concentrated. The residue was purified by flash chromatography (silica gel, hexane:acetic acid ethyl ester: 6:1) and resulted in the desired product (3.96 g).

¹H NMR (300 MHz, CDCl₃): δ: 8.70 (m, 1H), 7.05 (m, 1H), 4.36 (q, 2H), 1.35 (t, 3H)

### 3c) 2-Fluoro-4-methoxycarbonylmethylsulfanyl-5-nitro-benzoic acid ethyl ester

The nitrobenzoic acid (3b) (3.96 g) was dissolved in dichloromethane (75 ml), mixed with triethylamine (2.8 ml) and cooled to 0° C. After the addition of methyl thioglycolate (1.5 ml), the reaction mixture was stirred at 0-5° C. for 3.5 hours, and the solution was stored overnight in the refrigerator. The solution was concentrated, and the residue was purified by flash chromatography (silica gel, hexane:acetic acid ethyl ester: 6:1) and resulted in the desired product (3.86 g).

¹H NMR (300 MHz, CDCl₃): δ: 8.82 (d, 1H), 7.19 (d, 1H), 4.35 (q, 2H), 3.72 (s, 3H), 3.70 (s, 2H), 1.35 (t, 3H)

### 3d) 7-Fluoro-3-oxo-3,4-dihydro-2H-benzo[1,4]thiazine-6-carboxylic acid ethyl ester

The compound (3c) (3.86 g) was dissolved in acetic acid (142 ml), mixed with iron powder (6.8 g) and stirred at 60° C. for 4 hours. The reaction mixture was filtered through silica gel, rinsed again with methanol, and the filtrate was partially concentrated. Water and acetic acid ethyl ester were added, and the phases were separated. The aqueous phase was extracted once more with acetic acid ethyl ester. The combined organic phases were washed four times with water, dried over magnesium sulfate, filtered and concentrated, and resulted in the desired product (3.11 g).

¹H NMR (300 MHz, d₆-DMSO): δ: 10.71 (s, 1H), 7.50-7.39 (m, 2H), 4.30 (q, 2H), 3.56 (s, 2H), 1.30 (t, 3H)

### 3e) 7-Fluoro-3-oxo-3,4-dihydro-2H-benzo[1,4]thiazine-6-carboxylic acid

The thiazine (3d) (3.11 g) was suspended in THF (37 ml), mixed with a 1 N solution of sodium hydroxide (37 ml) and stirred at room temperature overnight. The solution was acidified to a pH value of 3 with 1 N solution of hydrochloric acid and partially concentrated. The produced precipitate was separated by filtration and washed with water. The solid was dried under reduced pressure (100 mbar, 40° C.) and resulted in the to desired product (2.49 g).

¹H NMR (300 MHz, d₆-DMSO): δ: 13.26 (bs, 1H), 10.72 (s, 1H), 7.48 (d, 1H), 7.38 (d, 1H), 3.57 (s, 2H)

### 3f) 7-Fluoro-6-hydroxymethyl-4H-benzo[1,4]thiazin-3-one

The thiazine carboxylic acid (3e) (2.49 g) was suspended in dry THF (80 ml), cooled to 0° C., mixed with triethylamine (1.8 ml) and isobutyl chloroformat (1.6 ml), and the reaction mixture was stirred for 30 minutes. The suspension was rapidly filtered through Celit into an ice cold solution of sodium borohydride (1.24 g) in water (24 ml). After 45 minutes, the solution was adjusted to a pH value of 1 with a 1 N solution of hydrochloric acid and extracted with acetic acid ethyl ester. The organic phase was washed with a saturated solution of sodium chloride, dried over magnesium sulfat, filtered and concentrated, and resulted in the desired product (2.29 g).

¹H NMR (300 MHz, d₆-DMSO): δ: 10.61 (s, 1H), 7.19 (d, 1H), 7.10 (d, 1H), 5.33 (m, 1H), 4.47 (d, 2H), 3.26 (s, 2H)

### 3g) 7-Fluoro-3-oxo-3,4-dihydro-2H-benzo[1,4]thiazine-6-carbaldehyde

The thiazinone (3f) (1.63 g) was dissolved in dichloromethane:THF 1:1 (138 ml), mixed with manganese dioxide (6.63 g) and stirred for 2 days at room temperature. Additional manganese dioxide (3.32 g) was added and stirred for further 3 days. The suspension was filtered through Celite and rinsed again with THF. The filtrate was concentrated, and the residue was purified by flash chromatography (silica gel, hexane:acetic acid ethyl ester: 7:3) and resulted in the desired product (765 mg).

¹H NMR (300 MHz, d₆-DMSO): δ: 10.80 (s, 1H), 10.14 (s, 1H), 7.51 (d, 1H), 7.35 (d, 1H), 3.60 (s, 2H)

### 3h) Title Compound

The compound was prepared as in example 1k starting from the aldehyde (3g) in a yield of 93%.

¹H NMR (300 MHz, d₆-DMSO): δ: 10.53 (s, 1H), 8.65 (d, 1H), 7.87-7.82 (m, 2H), 7.68 (d, 1H), 7.58-7.53 (m, 1H), 7.18 (d, 1H), 7.09 (d, 1H), 5.49-5.38 (m, 1H), 5.33-5.21 (m, 1H), 3.95 (s, 3H), 3.69 (s, 2H), 3.45 (s, 2H), 3.05-2.90 (m, 2H), 2.65-2.55 (m, 2H), 2.45-2.29 (m, 1H), 2.23-2.03 (m, 2H), 1.88-1.74 (m, 2H), 1.42-1.16 (m, 3H)

### Example 4

### 6-({1-[2-Hydroxy-2-(3-methoxy-quinolin-5-yl)-ethyl]-piperidinis 4-ylamino}-methyl)-4H-benzo[1,4]thiazin-3-one (enantiomer 2)

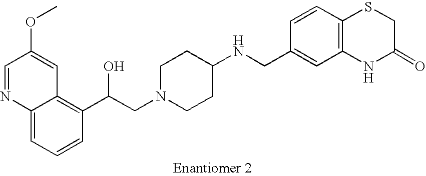

### 4a) (4-Formyl-2-nitro-phenylsulfanyl)acetic acid methyl ester

4-Chloro 3-nitrobenzaldehyde (10 g) was dissolved in DMF (100 ml), sodium hydride (2.35 g) was added thereto and stirred for 15 minutes at room temperature. Thereafter, methyl thioglycolate (3.45 ml) was added in drops and stirred for 5 hours at room temperature. The reaction mixture was diluted with water and extracted with acetic acid ethyl ester. The combined organic phases were washed twice with water, dried over sodium sulfate, filtered and concentrated. The crude product was purified by flash chromatography (silica gel, hexane:acetic acid ethyl ester: 2:1) and resulted in the desired product (5.5 g).

¹H NMR (300 MHz, CDCl₃): δ: 10.05 (s, 1H), 8.75 (d, 1H), 8.09 (dd, 1H), 7.68 (d, 1H), 3.84 (s, 2H), 3.81 (s, 3H)

### 4b) 3-Oxo-3,4-dihydro-2H-benzo[1,4]thiazine-6-carbaldehyde

The compound (4a) (5.5 g) was dissolved in acetic acid (115 ml), and iron powder (8.42 g) was added thereto. The reaction mixture was first stirred for 15 minutes at room temperature, and then for 3 hours at 50° C., and subsequently filtered through Decalit. The filter cake was washed with methanol, and the filtrate was concentrated. The residue was dissolved in a saturated solution of sodium hydrogencarbonate and extracted with acetic acid ethyl ester. The combined organic phases were dried over sodium sulfate, filtered and concentrated. The crude product was purified by flash chromatography (silica gel, hexane:acetic acid ethyl ester: 2:1) and resulted in the desired product (1 g).

¹H NMR (300 MHz, CDCl₃): δ: 10.18 (bs, 1H), 9.85 (s, 1H), 7.45-7.34 (m, 3H), 3.39 (s, 2H)

### 4c) Title Compound

The compound was prepared as in example 1k starting from the aldehyde (4b) in a yield of 80%.

¹H NMR (300 MHz, d₆-DMSO): δ: 10.49 (s, 1H), 8.65 (d, 1H), 7.87-7.82 (m, 2H), 7.68 (d, 1H), 7.58-7.53 (m, 1H), 7.23 (d, 1H), 6.97-6.91 (m, 2H), 5.46-5.36 (m, 1H), 5.28-5.20 (m, 1H), 3.95 (s, 3H), 3.62 (s, 2H), 3.43 (s, 2H), 3.04-2.98 (m, 2H), 2.65-2.55 (m, 2H), 2.43-2.28 (m, 1H), 2.20-2.00 (m, 2H), 1.85-1.71 (m, 2H), 1.40-1.16 (m, 3H)

### Example 5

### 2-{4-[(2,3-Dihydro-[1,4]dioxino[2,3-c]pyridin-7-ylmethyl)amino]-piperidin-1-yl}-1-(3-methoxy-quinolin-5-yl)-ethanol (enantiomer 2)

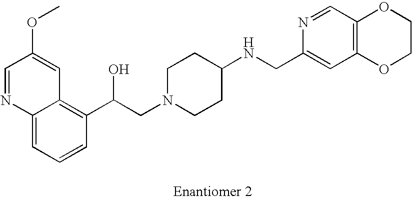

### 5a) 5-Benzyloxy-2-hydroxymethyl-pyran-4-one

To a solution of kojic acid (10.36 g) in warm methanol (135 ml), sodium methylate (4.3 g) was added in portions and then benzyl chloride (9.6 ml) was added in drops. The reaction mixture was heated overnight to 70° C., cooled down and poured on ice water. The solid was separated by filtration and dried, and resulted in the desired product (6.43 g).

¹H NMR (300 MHz, d₆-DMSO): δ: 8.18 (s, 1H), 7.44-7.32 (m, 5H), 6.33 (s, 1H), 5.71-5.66 (m, 1H), 4.95 (s, 2H), 4.30 (d, 2H)

### 5b) 5-Benzyloxy-2-hydroxymethyl-1H-pyridin-4-one

A suspension of the pyranone (5a) (6.43 g) in concentrated ammonia (67 ml) and ethanol (14 ml) was heated to reflux overnight. The solution was cooled, the solid was separated by filtration and dried, and resulted in the desired product (5.1 g).

¹H NMR (300 MHz, d₆-DMSO): δ: 11.17 (bs, 1H), 7.48-7.29 (m, 5H), 6.14 (bs, 1H), 5.59 (bs, 1H), 5.02 (s, 2H), 4.34 (s, 2H)

### 5c) (2,3-Dihydro-[1,4]dioxino[2,3-c]pyridin-7-yl)-methanol

To a solution of the pyridinone (5b) (12.6 g) in water (1.4 l), sodium hydroxide (4.36 g) and 10% palladium on charcoal (6.7 g) were added and hydrogenated for 2 days. The catalyst was separated by filtration, and the solution was lyophilized. The residue was dissolved in DMF (106 ml), potassium carbonate (18.13 g) and 1,2-dibromoethane (3.84 ml) were added thereto and heated to 100° C. overnight. After cooling the solution was concentrated, the residue was resuspended or redissolved, respectively, in water and extracted several times with acetic acid ethyl ester. The combined organic phases were dried over sodium sulfate, filtered and concentrated. The crude product was purified by flash chromatography (silica gel, dichloromethane:methanol: 9:1) and resulted in the desired product (1.49 g).

¹H NMR (300 MHz, d₆-DMSO): δ: 8.00 (s, 1H), 6.91 (s, 1H), 5.31-5.26 (m, 1H), 4.41 (d, 2H), 4.36-4.33 (m, 2H), 4.29-4.26 (m, 2H)

### 5d) 2,3-Dihydro-[1,4]dioxino[2,3-c]pyridine-7-carbaldehyde

To a solution of oxalyl chloride (2.2 ml) in dichloromethane (22 ml), a solution of dimethyl sulfoxide (DMSO) (2.2 ml) in dichloromethane (22 ml) was added thereto in drops at −78° C. and stirred for 15 minutes. Subsequently, a solution of the alcohol (5c) (1.49 g) in dichloromethane (16 ml) was added, stirred for 1 hour and then, a solution of triethylamine (8.7 ml) in dichloromethane (11 ml) was added thereto. After 20 minutes, the reaction mixture was heated to 0° C. and stirred for 30 minutes. Thereafter, water was added, the phases were separated, and the aqueous phase was extracted twice with dichloromethane. The combined organic phases were dried over sodium sulfate, filtered and concentrated. The crude product was purified by flash chromatography (silica gel, dichloromethane:methanol: 19:1) and resulted in the desired product (1.36 g).

¹H NMR (300 MHz, CDCl₃): δ: 9.91 (s, 1H), 8.24 (s, 1H), 7.45 (s, 1H), 4.33 (s, 4H)

### 5e) Title Compound

The compound was prepared as in example 1k starting from the aldehyde (5d) in a yield of 78%.

¹H NMR (300 MHz, d₆-DMSO): δ: 8.65 (d, 1H), 8.03 (s, 1H), 7.87-7.81 (m, 2H), 7.68 (d, 1H), 7.58-7.51 (m, 1H), 6.96 (s, 1H), 5.48-5.38 (m, 1H), 5.36-5.19 (m, 1H), 4.36-4.33 (m, 2H), 4.29-4.27 (m, 2H), 3.95 (s, 3H), 3.73 (s, 2H), 3.04-2.90 (m, 1H), 2.78-2.55 (m, 2H), 2.46-2.34 (m, 1H), 2.21-2.03 (m, 2H), 1.89-1.74 (m, 2H), 1.43-1.11 (m, 4H)

### Example 6

### 2-{4-[(2,3-Dihydro-benzo[1,4]dioxin-6-ylmethyl)-amino]-piperidin-1-yl}-1-(3-methoxy-quinolin-5-yl)-ethanol (enantiomer 2)

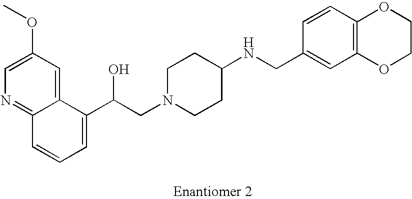

The compound was prepared as in example 1k starting from (2,3-dihydrobenzo[1,4]dioxine-6-carbaldehyde.

MS (EI): m/z: 450 [M+H]⁺

### Example 7

### 6-({1-[2-Hydroxy-2-(3-methoxy-quinolin-5-yl)-ethyl]piperidin-4-ylamino}-methyl)-4H-benzo[1,4]oxazin-3-one (enantiomer 1)

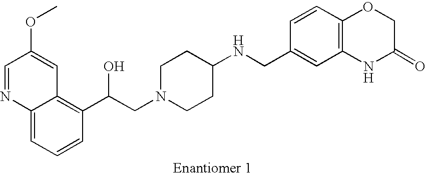

### 7a) 3-Methoxy-5-oxiranyl-quinoline (enantiomer 1)

The compound was synthesized as described in the examples 1d to if. Instead of the AD mix alpha, AD mix beta was used for the preparation of the diol (1d).

### 7b) {1-[2-Hydroxy-2-(3-methoxy-quinolin-5-yl)-ethyl]-piperidin-4-yl}-carbamic acid tert-butyl ester (enantiomer 1)

The title compound was prepared as in example 1g starting from the epoxide (7a) in a yield of 56%.

¹H NMR (300 MHz, CDCl₃): δ: 8.56 (d, 1H), 7.92 (m, 1H), 7.64 (m, 1H), 7.48-7.43 (m, 1H), 5.72 (bs, 1H), 4.51 (m, 1H), 3.85 (s, 3H), 3.60-3.46 (m, 1H), 3.29-3.26 (m, 2H), 2.84-2.73 (m, 2H), 2.67-2.40 (m, 2H), 2.09-1.95 (m, 2H), 1.84-1.64 (m, 2H), 1.38 (s, 9H)

### 7c) 2-(4-Amino-piperidin-1-yl)-1-(3-methoxy-quinolin-5-yl)-ethanol (enantiomer 1)

The compound was prepared as in example 1h starting from the Boc-amine (7b) in a yield of 63%.

¹H NMR (300 MHz, d₆-DMSO): δ: 8.66-8.63 (m, 1H), 7.87-7.82 (m, 2H), 7.67 (m, 1H), 7.58-7.51 (m, 1H), 5.43-5.39 (m, 1H), 5.26-5.23 (m, 1H), 3.95 (s, 3H), 2.99-2.85 (m, 2H), 2.62-2.55 (m, 1H), 2.19-1.99 (m, 2H), 1.72-1.57 (m, 2H)

### 7d) Title Compound

The compound was prepared as in example 1k starting from the amine (7c) and the aldehyde (1j) in a yield of 86%.

¹H NMR (300 MHz, d₆-DMSO): δ: 10.78 (s, 1H), 8.66 (d, 1H), 7.96 (s, 1H), 7.88-7.84 (m, 2H), 7.70-7.68 (m, 1H), 7.59-7.54 (m, 1H), 7.00-6.91 (m, 3H), 5.75 (s, 1H), 5.49-5.46 (m, 1H), 4.56 (s, 2H), 3.96 (s, 3H), 2.82 (s, 2H), 3.12-3.03 (m, 2H), 2.72-2.60 (m, 2H), 2.30-2.10 (m, 2H), 2.00-1.85 (m, 2H), 1.58-1.36 (m, 3H)

### Example 8

### 7-Fluoro-6-({1-[2-hydroxy-2-(3-methoxy-quinolin-5-yl)-ethyl]-piperidin-4-ylamino}-methyl)-4H-benzo[1,4]thiazin-3-one (enantiomer 1)

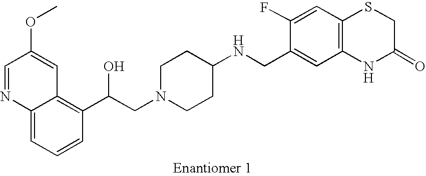

The compound was prepared as in example 7d starting from the aldehyde (3g) in a yield of 78%.

¹H NMR (300 MHz, d₆-DMSO): δ: 10.53 (s, 1H), 8.65 (d, 1H), 7.87-7.82 (m, 2H), 7.68 (d, 1H), 7.58-7.53 (m, 1H), 7.18 (d, 1H), 7.09 (d, 1H), 5.49-5.38 (m, 1H), 5.33-5.21 (m, 1H), 3.95 (s, 3H), 3.69 (s, 2H), 3.45 (s, 2H), 3.05-2.90 (m, 2H), 2.65-2.55 (m, 2H), 2.45-2.29 (m, 1H), 2.23-2.03 (m, 2H), 1.88-1.74 (m, 2H), 1.42-1.16 (m, 3H)

### Example 9

### 6-({1-[2-Hydroxy-2-(3-methoxy-quinolin-5-yl)-ethyl]-piperidin-4-ylamino}-methyl)-4H-benzo[1,4]thiazin-3-one (enantiomer 1)

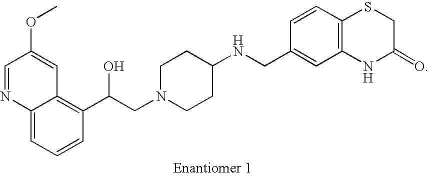

The compound was prepared as in example 7d starting from the aldehyde (4b) in a yield of 73%.

¹H NMR (300 MHz, d₆-DMSO): δ: 10.49 (s, 1H), 8.65 (d, 1H), 7.87-7.82 (m, 2H), 7.68 (d, 1H), 7.58-7.53 (m, 1H), 7.23 (d, 1H), 6.97-6.91 (m, 2H), 5.46-5.36 (m, 1H), 5.28-5.20 (m, 1H), 3.95 (s, 3H), 3.62 (s, 2H), 3.43 (s, 2H), 3.04-2.98 (m, 2H), 2.65-2.55 (m, 2H), 2.43-2.28 (m, 1H), 2.20-2.00 (m, 2H), 1.85-1.71 (m, 2H), 1.40-1.16 (m, 3H)

### Example 10

### 6-({1-[2-Hydroxy-2-(3-methoxy-quinolin-5-yl)-ethyl]-piperidin-4-ylamino}-methyl)-4H-pyrido[3,2-b][1,4]thiazin-3-one (enantiomer 1)

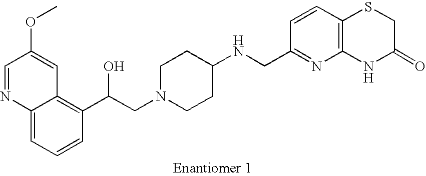

The compound was prepared as in example 7d starting from the aldehyde (2h) in a yield of 83%.

¹H NMR (300 MHz, d₆-DMSO): δ: 10.85 (s, 1H), 8.65 (d, 1H), 7.87-7.82 (m, 2H), 7.74-7.67 (m, 2H), 7.58-7.51 (m, 1H), 7.11-7.05 (m, 1H), 5.50-5.36 (m, 1H), 5.30-5.19 (m, 1H), 3.95 (m, 1H), 3.72 (s, 2H), 3.53 (s, 2H), 3.02-2.87 (m, 2H), 2.65-2.54 (m, 2H), 2.44-2.32 (m, 1H), 2.21-2.02 (m, 2H), 1.86-1.70 (m, 3H), 1.42-1.13 (m, 3H)

### Example 11

### 2-{4-[(2,3-Dihydro-[1,4]dioxino[2,3-c]pyridin-7-ylmethyl)amino]-piperidin-1-yl}-1-(3-methoxy-quinolin-5-yl)-ethanol (enantiomer 1)

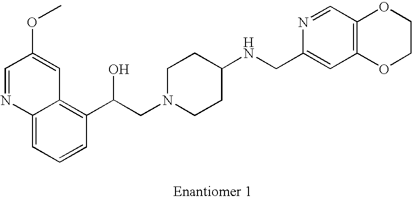

The compound was prepared as in example 7d starting from the aldehyde (5d) in a yield of 78%.

¹H NMR (300 MHz, d₆-DMSO): δ: 8.65 (d, 1H), 8.03 (s, 1H), 7.87-7.81 (m, 2H), 7.68 (d, 1H), 7.58-7.51 (m, 1H), 6.96 (s, 1H), 5.48-5.38 (m, 1H), 5.36-5.19 (m, 1H), 4.36-4.33 (m, 2H), 4.29-4.27 (m, 2H), 3.95 (s, 3H), 3.73 (s, 2H), 3.04-2.90 (m, 1H), 2.78-2.55 (m, 2H), 2.46-2.34 (m, 1H), 2.21-2.03 (m, 2H), 1.89-1.74 (m, 2H), 1.43-1.11 (m, 4H)

### Example 12

### 2-{4-[(2,3-Dihydro-benzo[1,4]dioxin-6-ylmethyl)-amino]-piperidin-1-yl}-1-(3-methoxy-quinolin-5-yl)-ethanol (enantiomer 1)

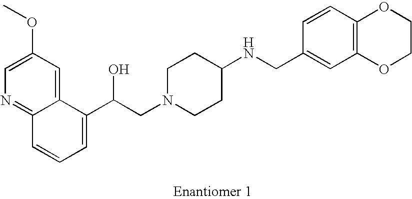

The compound was prepared as in example 7d starting from 2,3-dihydrobenzo[1,4]dioxine-6-carbaldehyde.

MS (EI): m/z: 450 [M+H]⁺

### Example 13

### 2-{1-[2-Hydroxy-2-(3-methoxy-quinolin-5-yl)-ethyl]-piperidin-4-ylamino}-N-pyridin-2-yl-acetamide

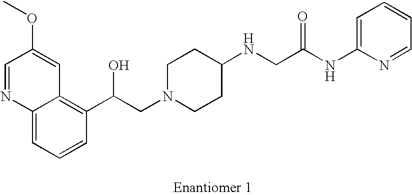

### 13a) 2-Chloro-N-pyridin-2-yl-acetamide

Chloro acetylchloride (1.8 ml) was added in drops to an ice cold solution of 2-aminopyridine (1.88 g) in THF:pyridine (20 ml, 1:1). After stirring for 3 hours at room temperature, the solution was poured into water and extracted with acetic acid ethyl ester. The combined organic phases were dried over magnesium sulfate, filtered and concentrated. The residue was recrystallized from acetic acid ethyl ester:ether and resulted in the desired product (3.4 g).

MS (EI): m/z: 171 [M+H]⁺

### 13b) Title Compound

To a solution of the amine (7c) (0.05 g) in DMF (1 ml), the chloride (13a) (0.034 g) and sodium carbonate (0.05 g) were added and stirred for 2 hours at 60° C. After concentrating the reaction mixture, the crude product was purified by flash chromatography (silica gel, dichloromethane:methanol: 9:1) and resulted in the desired product.

MS (EI): m/z: 436 [M+H]+

### Example 14

### 2-{-4-[(2,3-Dihydro-benzo[1,4]dioxin-6-ylmethyl)-amino]-piperidin-1-yl}-1-(3-methoxy-quinolin-5-yl)-ethanol (racemate)

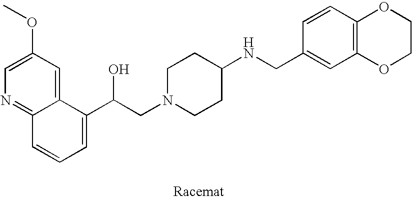

### 14a) 3-Methoxy-quinoline-5-carbaldehyde

To a solution of the bromide (1b) (2.68 g) in ether (37 ml) and THF (37 ml), n-butyl lithium (9.3 ml, 2.5 M in hexane) was added at −78° C., stirred for 30 minutes and quenched with DMF (5 ml). After 15 minutes, ethanol (8 ml) and a solution of ammonium chloride (50 ml) were added and heated to room temperature. The aqueous phase was extracted with acetic acid ethyl ester. The combined organic phases were washed with a saturated solution of sodium chloride, dried over magnesium sulfat, filtered and concentrated. The residue was purified by flash chromatography (silica gel, hexane:acetic acid ethyl ester: 2:1, 1:2) and resulted in the desired product (1.06 g).

MS (EI): m/z: 188 [M+H]⁺

### 14b) 3-Methoxy-5-oxiranyl-quinoline

To a solution of the aldehyde (14a) (1.06 g) in acetonitrile (17.6 ml), 9 drops of water, trimethylsulfonium iodide (1.19 g) and potassium hydroxide (2.25 g) were added and heated to 60° C. for 20 minutes. After cooling, the solution was filtered, the filtrate was diluted with water (10 ml) and concentrated. The residue was extracted with acetic acid ethyl ester and the combined organic phases were concentrated. The residue was purified by flash chromatography (silica gel, hexane:acetic acid ethyl ester: 2:1, 1:1) and resulted in the desired product (1 g).

¹H NMR (300 MHz, CDCl₃): δ: 8.64 (d, 1H), 7.94 (dd, 1H), 7.59 (d, 1H), 7.48-7.39 (m, 2H), 4.30 (m, 1H), 3.91 (s, 3H), 3.22 (dd, 1H), 2.81 (dd, 1H)

### 14c) {1-[2-Hydroxy-2-(3-methoxy-quinolin-5-yl)-ethyl]-piperidin-4-yl}-carbamic acid tert-butyl ester (racemate)

The compound was prepared as in example 1g starting from the epoxide (14b) in a yield of 65%.

¹H NMR (300 MHz, CDCl₃): δ: 8.56 (d, 1H), 7.92 (m, 1H), 7.64 (m, 1H), 7.48-7.43 (m, 1H), 5.72 (bs, 1H), 4.51 (m, 1H), 3.85 (s, 3H), 3.60-3.46 (m, 1H), 3.29-3.26 (m, 2H), 2.84-2.73 (m, 2H), 2.67-2.40 (m, 2H), 2.09-1.95 (m, 2H), 1.84-1.64 (m, 2H), 1.38 (s, 9H)

### 14d) 2-(4-Amino-piperidin-1-yl)-1-(3-methoxy-quinolin-5-yl)-ethanol (racemate)

The compound was prepared as in example 1h starting from the Boc-amine (14c) in a yield of 78%.

MS (EI): m/z: 302 [M+H]⁺

### 14e) Title Compound

The title compound was prepared as in example 1k starting from the amine (14d) and 2,3-dihydro-benzo[1,4]dioxine-6-carbaldehyde in a yield of 50%.

MS (EI): m/z: 450 [M+H]⁺

### Example 15

### 2-{4-[(Benzo[1,2,5]thiadiazol-5-ylmethyl)-amino]-piperidin-1-yl}-1-(3-methoxy-quinolin-5-yl)-ethanol (racemate)

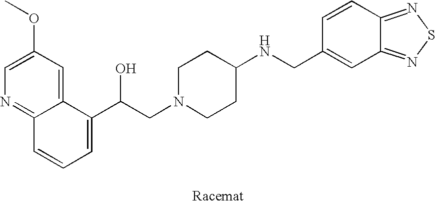

The compound was prepared as in example 14e starting from benzo[1,2,5]thiadiazole-5-carbaldehyde.

MS (EI): m/z: 450 [M+H]⁺

### Example 16

### 1-(3-Methoxy-quinolin-5-yl)-2-{4-[(2-methyl-benzofuran-5-ylmethyl)-amino]-piperidin-1-yl}-ethanol (racemate)

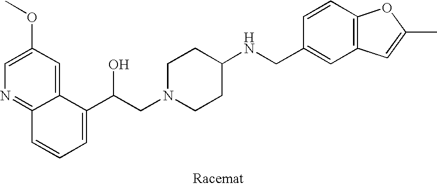

### 16a) 4-Prop-2-ynyloxy benzaldehyde

To a suspension of 4-hydroxy benzaldehyde (5.91 g) in toluene (80 ml), potassium carbonate (87.4 g) and propargyl bromide (8 ml, 80% solution in toluene) were added and heated to 100° C. for 7 hours. Then, the suspension was filtered, and the filtrate was concentrated. The residue was purified by flash chromatography (silica gel, hexane:acetic acid ethyl ester: 3:1), and resulted in the desired product (5 g).

MS (EI): m/z: 161 [M+H]⁺

### 16b) 2-Methyl-benzofuran-5-carbaldehyde

PEG 300 (40 ml) was heated to 220° C. A solution of the propynyl aldehyde (16a) (5 g) in PEG 300 (10 ml) was added and stirred at 220° C. for 90 minutes. After cooling, the reaction mixture was poured on ice (200 g) and extracted with dichloromethane:ether (1:1, 2×300 ml). The combined organic phases were concentrated. The residue was purified by flash chromatography (silica gel, hexane:acetic acid ethyl ester: 9:1) and resulted in the desired product (2 g).

¹H NMR (300 MHz, CDCl₃): δ: 9.93 (s, 1H), 7.91 (s, 1H), 7.67 (dd, 1H), 7.40 (d, 1H), 6.38 (s, 1H), 2.39 (s, 3H)

### 16c) Title Compound

The compound was prepared as in example 14e starting from the aldehyde (16b).

MS (EI): m/z: 446 [M+H]⁺

### Example 17

### 1-(3-Methoxy-quinolin-5-yl)-2-{4-[(quinoxalin-2-ylmethyl)amino]-piperidin-1-yl}-ethanol (racemate)

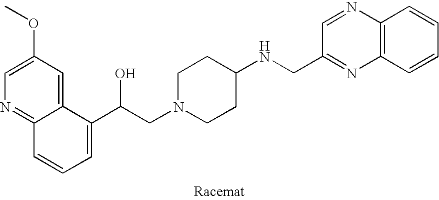

### 17a) Quinoxaline-2-carbaldehyde

To a solution of selenium dioxide (12 g) in dioxane (120 ml) and water (5 ml) boiling under reflux, a solution of 2-methyl quinoxaline (10 g) in dioxane (20 ml) was added in drops and subsequently heated to reflux for 4 hours. After cooling, the suspension was filtered through silica gel, and the filtrate was concentrated. The residue was purified by flash chromatography (silica gel, hexane:acetic acid ethyl ester: 2:1) and resulted to in the desired product (8 g).

MS (EI): m/z: 159 [M+H]⁺

### 17b) Title Compound

The compound was prepared as in example 14e starting from the aldehyde (17a).

MS (EI): m/z: 445 [M+H]⁺

### Example 18

### 2-[4-((E)-3-Furan-2-yl-allylamino)-piperidin-1-yl]-1-(3-methoxy-quinolin-5-yl)-ethanol (racemate)

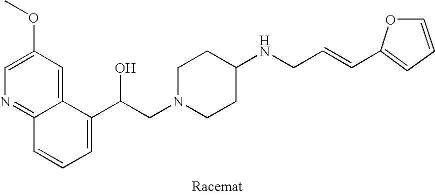

The compound was prepared as in example 14e starting from (E)-3-furan-2-yl-propenal.

MS (EI): m/z: 408 [M+H]⁺

### Example 19

### 2-{4-[(Benzofuran-2-ylmethyl)-amino]-piperidin-1-yl}-1-(3-methoxy-quinolin-5-yl)-ethanol (racemate)

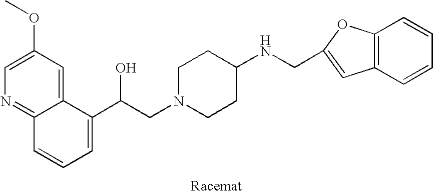

The compound was prepared as in example 14e starting from benzofuran-2-carbaldehyde.

MS (EI): m/z: 432 [M+H]⁺

### Example 20

### 1-(3-Methoxy-quinolin-5-yl)-2-[4-(2-phenoxy-ethylamino)piperidin-1-yl]-ethanol (racemate)

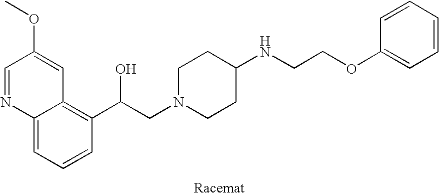

The compound was prepared as in example 14e starting from phenoxy acetaldehyde (according to Syn. Lett., vol. 11, 2004, p. 2010).

MS (EI): m/z: 422 [M+H]⁺

### Example 21

### 5-({1-[2-Hydroxy-2-(3-methoxy-quinolin-5-yl)-ethyl]-piperidin-4-ylamino}-methyl)-3H-benzoxazol-2-one (racemate)

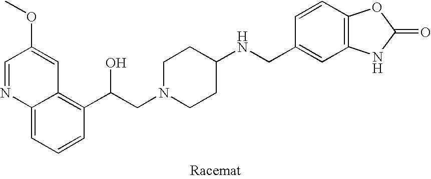

### 21a) 4-[1,3]Dioxan-2-yl-2-nitro-phenol

To a solution of 4-hydroxy-3-nitro benzaldehyde (5.54 g) in toluene (110 ml), 1,3-propandiol (3.80 g) and p-toluene sulfonic acid (0.11 g) were added and heated to reflux for 3 hours. After cooling, the solution was washed with a saturated solution of sodium hydrogencarbonate, and the aqueous phase was reextracted with ether. The combined organic phases were washed with a saturated solution of sodium chloride, dried over magnesium sulfate, filtered and concentrated, and resulted in the desired product (3.5 g).

¹H NMR (300 MHz, CDCl₃): δ: 10.63 (s, 1H), 8.26 (s, 1H), 7.73 (d, 1H), 7.17 (d, 1H), 5.49 (s, 1H), 4.32-4.26 (m, 2H), 4.04-3.96 (m, 2H), 1.51-1.46 (m, 2H)

### 21b) 2-Amino-4-[1,3]dioxan-2-yl-phenol

To a solution of the nitrophenol (21a) (2 g) in THF, Raney nickel was added and stirred for 5 hours under a hydrogen gas atmosphere. The reaction mixture was filtered, and the filter cake was washed with acetic acid ethyl ester. The filtrate was concentrated and resulted in the desired product (1.66 g).

MS (EI): m/z: 196 [M+H]⁺

### 21c) 5-[1,3]Dioxan-2-yl-3H-benzoxazol-2-one

To a solution of the aminophenol (21b) (1.66 g) in dichloromethane (40 ml), triethylamine (1.72 ml) and triphosgene (3.33 g) were added at 0° C. and stirred for 1.5 hours. The solution was concentrated and the residue was purified by flash chromatography (silica gel, acetic acid ethyl ester:hexane: 1:1) and resulted in the desired product (1.5 g).

MS (EI): m/z: 222 [M+H]⁺

### 21d) 2-Oxo-2,3-dihydro-benzoxazole-5-carbaldehyde

To a solution of the benzoxazole (21c) (1.5 g) in methanol (20 ml), a 3 M solution of hydrochloric acid (2.5 ml) was added and stirred for 2 hours. Thereafter, the solution was partially concentrated and extracted with acetic acid ethyl ester. The combined organic phases were washed with a saturated solution of sodium hydrogencarbonate, dried over magnesium sulfate, filtered and concentrated and resulted in the desired product (0.84 g).

¹H NMR (300 MHz, d₆-DMSO): δ: 12.00 (bs, 1H), 9.96 (s, 1H), 7.73 (d, 1H), 7.54-7.50 (m, 2H)

### 21e) Title Compound

The title compound was prepared as in example 14e starting from the aldehyde (21d).

MS (EI): m/z: 449 [M+H]⁺

### Example 22

### 2-{4-[(2,3-Dihydro-[1,4]dioxino[2,3-c]pyridin-7-ylmethyl)amino]-piperidin-1-yl}-1-(3-methoxy-quinolin-5-yl)-ethanol (racemate)

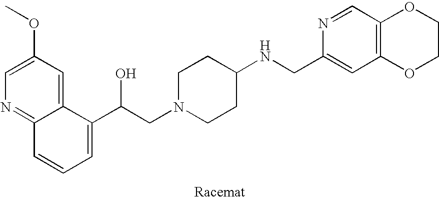

The compound was prepared as in example 14e starting from the aldehyde (5d).

MS (EI): m/z: 451 [M+H]⁺

### Example 23

### 2-{4-[(3,4-Dihydro-2H-benzo[b][1,4]dioxepin-7-ylmethyl)amino]-piperidin-1-yl}-1-(3-methoxy-quinolin-5-yl)-ethanol (racemate)

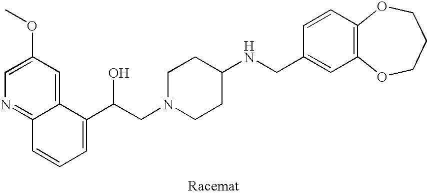

### 23a) 3,4-Dihydro-2H-benzo[b][1,4]dioxepine-7-carbaldehyd

To a solution of 3,4-dihydroxy benzaldehyde (1.67 g) and potassium carbonate (3.34 g) in acetonitrile (15 ml), 1,3-dibromopropane (1.36 ml) was added and heated to reflux. Thereafter, the reaction mixture was filtered and rinsed again with ether. The filtrate was concentrated and the residue was purified by flash chromatography (silica gel, hexane:acetic acid ethyl ester: 4:1) and resulted in the desired product (1.5 g).

¹H NMR (300 MHz, CDCl₃): δ: 9.86 (s, 1H), 7.50-7.43 (m, 2H), 7.08-7.04 (m, 1H), 4.39-4.21 (m, 4H), 2.31-2.23 (m, 2H)

### 23b) Title Compound

The compound was prepared as in example 14e starting from the aldehyde (23a).

MS (ED: m/z: 464 [M+H]⁺

### Example 24

### 1-(3-Methoxy-quinolin-5-yl)-2-[4-((E)-3-phenyl-allylamino)piperidin-1-yl]-ethanol (racemate)

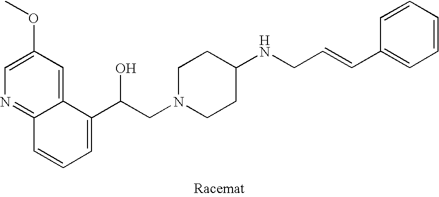

The compound was prepared as in example 14e starting from cinnamic aldehyde.

MS (EI): m/z: 418 [M+H]⁺

### Example 25

### 1-(3-Methoxy-quinolin-5-yl)-2-{4-[(1-methyl-1H-indol-2-ylmethyl)-amino]-piperidin-1-yl}-ethanol (racemate)

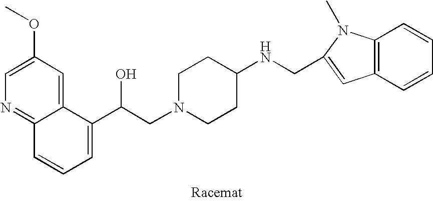

The compound was prepared as in example 14e starting from 1-methyl-1H-indol-2-carbaldehyde.

MS (EI): m/z: 445 [M+H]⁺

### Example 26

### 1-(3-Methoxy-quinolin-5-yl)-2-[4-(3-phenyl-propylamino)piperidin-1-yl]-ethanol (racemate)

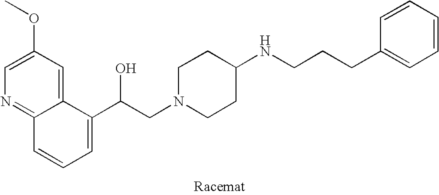

The compound was prepared as in example 14e starting from 3-phenyl propionaldehyde.

MS (EI): m/z: 420 [M+H]⁺

### Example 27

### 7-({1-[2-Hydroxy-2-(3-methoxy-quinolin-5-yl)-ethyl]-piperidin-4-ylamino}-methyl)-4H-benzo[1,4]oxazin-3-one (racemate)

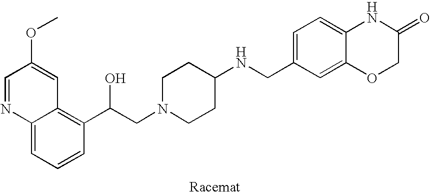

### 27a) (5-Formyl-2-nitro-phenoxy)acetic acid ethyl ester

To a solution of 3-hydroxy-4-nitro benzaldehyde in DMF (100 ml), chloroacetic acid ethyl ester (7 ml) and potassium carbonate (10 g) were added and heated for 2 hours to 50° C. Water was added to the reaction mixture, and the reaction mixture was extracted with acetic acid ethyl ester:ether. The organic phase was concentrated and the residue was purified by flash chromatography (silica gel, hexane:acetic acid ethyl ester: 4:1, 3:1, 2:1, 1:1) and resulted in the desired product (14.2 g).

¹H NMR (300 MHz, CDCl₃): δ: 10.04 (s, 1H), 7.98 (d, 1H), 7.62 (d, 1H), 7.50 (s, 1H), 4.87 (s, 2H), 4.29 (q, 2H), 1.31 (t, 3H)

### 27b) 7-Hydroxymethyl-4H-benzo[1,4]oxazin-3-one

A suspension of the nitrobenzaldehyd (27a) (7 g) in acetic acid (200 ml) was mixed with iron powder (15.4 g) and heated to reflux. Thereafter, the reaction mixture was filtered through Celit and rinsed again with acetic acid. The filtrate was concentrated, and the residue was resuspended or redissolved, respectively, in acetic acid ethyl ester and washed with a saturated solution of sodium hydrogencarbonate. The organic phase was dried over sodium sulfate, filtered and concentrated. The crude product was purified by flash chromatography (silica gel, hexane:acetic acid ethyl ester: 1:1) and resulted in the desired product (1.5 g).

¹H NMR (300 MHz, d₆-DMSO): δ: 10.64 (s, 1H), 6.88-6.81 (m, 3H), 5.13-5.09 (m, 1H), 4.54 (s, 2H), 4.38 (d, 2H)

### 27c) 3-Oxo-3,4-dihydro-2H-benzo[1,4]oxazine-7-carbaldehyde

A solution of the alcohol (27b) (1.5 g) in THF:dichloromethane (150 ml, 1:1) was mixed with manganese dioxide (7.3 g) and stirred for 1.5 hours. Then, the reaction mixture was filtered through Celit, rinsed again with THF and the filtrate was concentrated. The residue was treated with ether, the produced solid was separated by filtration and resulted in the desired product (1 g).

¹H NMR (300 MHz, d₆-DMSO): δ: 9.83 (s, 1H), 7.54 (d, 1H), 7.43 (s, 1H), 7.06 (d, 1H), 4.68 (s, 2H)

### 27d) Title Compound

The compound was prepared as in example 14e starting from the aldehyde (27c).

MS (EI): m/z: 464 [M+H]⁺

### Example 28

### 6-({1-[2-Hydroxy-2-(3-methoxy-quinolin-5-yl)-ethyl]-piperidin-4-ylamino}-methyl)-4H-benzo[1,4]oxazin-3-one (racemate)

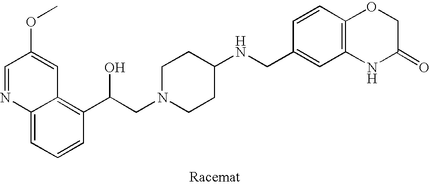

The title compound was prepared as in example 14e starting from the aldehyde (1j).

¹H NMR (300 MHz, d₆-DMSO): δ: 10.78 (s, 1H), 8.66 (d, 1H), 7.96 (s, 1H), 7.88-7.84 (m, 2H), 7.70-7.68 (m, 1H), 7.59-7.54 (m, 1H), 7.00-6.91 (m, 3H), 5.75 (s, 1H), 5.49-5.46 (m, 1H), 4.56 (s, 2H), 3.96 (s, 3H), 2.82 (s, 2H), 3.12-3.03 (m, 2H), 2.72-2.60 (m, 2H), 2.30-2.10 (m, 2H), 2.00-1.85 (m, 2H), 1.58-1.36 (m, 3H)

### Example 29

### 6-({1-[2-Hydroxy-2-(3-methoxy-quinolin-5-yl)-ethyl]-piperidin-4-ylamino}-methyl)-4H-pyrido[3,2b][1,4]oxazin-3-one (racemate)

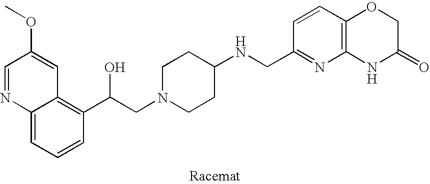

The title compound was prepared as in example 14e starting from 3-oxo-3,4-dihydro-2H-pyrido[3,2-b][1,4]oxazine-6-carbaldehyde (this compound was prepared according to WO 2006/021 448).

MS (EI): m/z: 464 [M+H]⁺

### Example 30

### 1-(3-Methoxy-quinolin-5-yl)-2-[4-((E)-3-pyridin-2-yl-allylamino)-piperidin-1-yl]-ethanol (racemate)

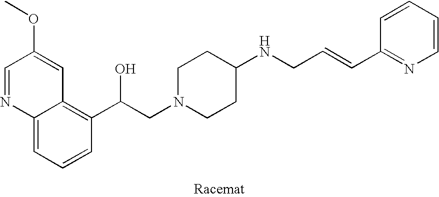

The title compound was prepared as in example 14e starting from (E)-3-pyridin-2-ylpropenal (this compound was prepared according to WO 2006/021 448).

MS (EI): m/z: 419 [M+H]⁺

### Example 31

### 2-{4-[(E)-3-(2,5-Difluoro-phenyl)-allylamino]-piperidin-1-yl}-1-(3-methoxy-quinolin-5-yl)-ethanol (racemate)

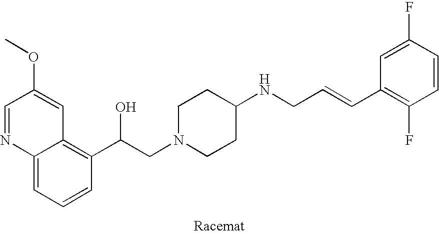

The title compound was prepared as in example 14e starting from (E)-3-(2,5-difluorophenyl) propenal (this compound was prepared according to WO 2004/087 647).

MS (EI): m/z: 454 [M+H]⁺

### Example 32

### 1-(3-Methoxy-quinolin-5-yl)-2-{4-[(naphthalen-2-ylmethyl)amino]-piperidin-1-yl}-ethanol

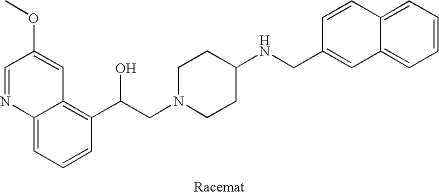

The title compound was prepared as in example 14e starting from naphthalene-2-carbaldehyde.

MS (EI): m/z: 442 [M+H]⁺

### Example 33

### 3-Oxo-3,4-dihydro-2H-benzo[1,4]oxazine-6-carboxylic acid {1-[2-hydroxy-2-(3-methoxy-quinolin-5-yl)-ethyl]-piperidin-4-yl}amide (racemate)

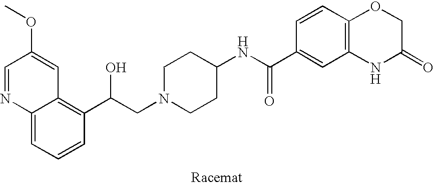

To a solution of the amine (14d) (180 mg) in a mixture of dichloromethane (6 ml) and DMF (1 ml), 3-oxo-3,4-dihydro-2H-benzo[1,4]oxazine-6-carboxylic acid (116 mg), EDC (112 mg) and HOBT (98 mg) were added at room temperature. After stirring for 12 hours at room temperature, the solution was concentrated. The residue was purified by flash chromatography (silica gel, 2-3% methanol in dichloromethane+1 ammonium hydroxide), and resulted in the desired product (111 mg).

MS (EI): m/z: 477 [M+H]⁺

### Example 34

### 3-Oxo-3,4-dihydro-2H-benzo[1,4]thiazine-6-carboxylic acid {1-[2-hydroxy-2-(3-methoxy-quinolin-5-yl)-ethyl]-piperidin-4-yl}amide (racemate)

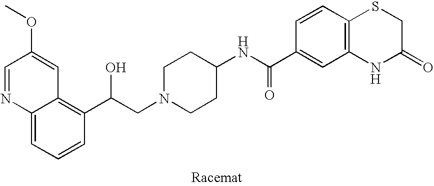

The title compound was prepared as in example 33 starting from 3-oxo-3,4-dihydro-2H-benzo[1,4]thiazine-6-carboxylic acid.

MS (ED: m/z: 493 [M+H]⁺

### Example 35

### 3-Oxo-3,4-dihydro-2H-pyrido[3,2-b][1,4]thiazine-6-carboxylic acid {1-[2-hydroxy-2-(3-methoxy-quinolin-5-yl)-ethyl]-piperidin-4-yl}amide (racemate)

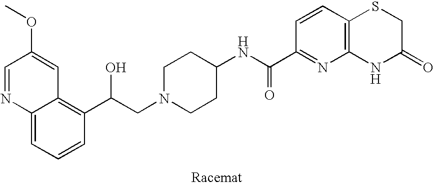

The title compound was prepared as in example 33 starting from 3-oxo-3,4-dihydro-2H-pyrido[3,2-b][1,4]thiazine-6-carboxylic acid (2f).

MS (EI): m/z: 494 [M+H]⁺

### Example 36

### 2,3-Dihydro-benzo[1,4]dioxine-6-carboxylic acid {1-[2-hydroxy-2-(3-methoxy-quinolin-5-yl)-ethyl]-piperidin-4-yl}amide (racemate)

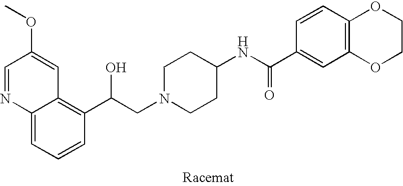

The title compound was prepared as in example 33 starting from 2,3-dihydrobenzo[1,4]dioxine-6-carboxylic acid.

MS (EI): m/z: 464 [M+H]⁺

### Example 37

### 3-Oxo-3,4-dihydro-2H-benzo[1,4]oxazine-6-sulfonic acid {1-[2-hydroxy-2-(3-methoxy-quinolin-5-yl)-ethyl]-piperidin-4-yl}amide (racemate)

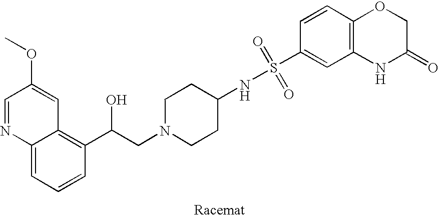

To a solution of the amine (14d) (0.3 g) in dry dichloromethane (15 ml), triethylamine (0.21 ml) and 3-oxo-3,4-dihydro-2H-benzo[1,4]oxazine-6-sulfonylchloride (0.27 g) were added at room temperature. After stirring for 24 hours at room temperature, the reaction mixture was concentrated. The residue was purified by flash chromatography (silica gel, chloroform:methanol: 9:1+5% ammonium hydroxide) and resulted in the desired product (0.15 g).

MS (EI): m/z: 513 [M+H]⁺

### Example 38

### 3-Oxo-3,4-dihydro-2H-benzo[1,4]thiazine-6-sulfonic acid {1-[2-hydroxy-2-(3-methoxy-quinolin-5-yl)ethyl]-piperidin-4-yl}amide (racemate)

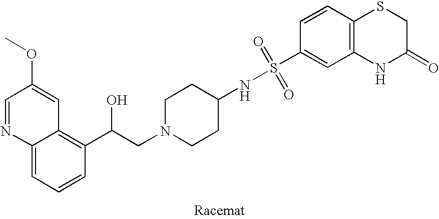

The title compound was prepared as in example 37 starting from 3-oxo-3,4-dihydro-2H-benzo[1,4]thiazine-6-sulfonylchloride.

MS (EI): m/z: 529 [M+H]⁺

### Example 39

### 2,3-Dihydro-benzo[1,4]dioxine-6-sulfonic acid {1-[2-hydroxy-2-(3-methoxy-quinolin-5-yl)-ethyl]-piperidin-4-yl}amide (racemate)

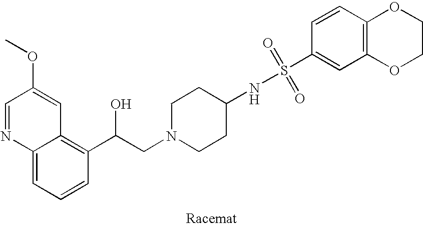

The title compound was prepared as in example 37 starting from 2,3-dihydrobenzo[1,4]dioxine-6-sulfonylchloride.

MS (EI): m/z: 500 [M+H]⁺

### Example 40

### 2-{4-[(2,3-Dihydro-benzo[1,4]dioxin-6-ylmethyl)-amino]-3-fluoro-piperidin-1-yl}-1-(3-methoxy-quinolin-5-yl)-ethanol (enantiomer 1)

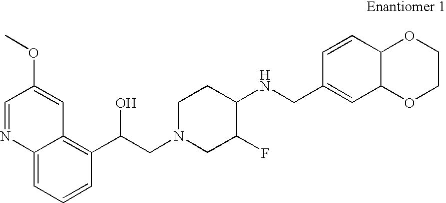

### 40a) 4-Oxo-piperidine-1-carboxylic acid tert-butyl ester

4-Piperidone hydrochloride hydrate (15.00 g) was dissolved in an 1 N solution of sodium hydroxide (102 ml), water (102 ml) and dioxane (102 ml). A solution of Boc anhydride (23.44 g) in dioxane (102 ml) was added in drops at room temperature, and the reaction mixture was stirred overnight at room temperature. The solution was partially concentrated and several times extracted with acetic acid ethyl ester. The combined organic phases were washed with a saturated solution of sodium chloride, dried over magnesium sulfate, concentrated and resulted in the desired product (19.27 g).

MS (EI): m/z: 200 [M+H]⁺

### 40b) 4-Trimethylsilanyloxy-3,6-dihydro-2H-pyridine-1-carboxylic acid tert-butyl ester

To a solution of the ketone (40a) (10.12 g) in DMF (20 ml), trimethylsilyl chloride (7.8 ml) and triethylamine (17 ml) were added and heated to 80° C. overnight. After cooling, the DMF was removed under reduced pressure, the residue was resuspended or redissolved, respectively, in a saturated solution of sodium hydrogencarbonate and extracted with hexane. The organic phase was dried over magnesium sulfate, filtered and concentrated. The residue was purified by flash chromatography (silica gel, hexane:acetic acid ethyl ester: 9:1), and resulted in the desired product (10.5 g).

MS (EI): m/z: 272 [M+H]⁺

### 40c) 3-Fluoro-4-oxo-piperidine-1-carboxylic acid tert-butyl ester

To a solution of the silylenol ether (40b) (10.5 g) in acetonitrile (420 ml), Selectfluor (15.1 g) was added and stirred for 75 minutes at room temperature. A saturated solution of sodium chloride was added, and the acetonitrile was removed under reduced pressure. The residue was extracted with acetic acid ethyl ester and the organic phase was dried over sodium sulfate, filtered and concentrated. The residue was purified by flash chromatography (aluminium oxide III, acetic acid ethyl ester, acetic acid ethyl ester:methanol: 9:1), and resulted in the desired product (8.5 g).

MS (EI): m/z: 218 [M+H]⁺

### 40d) 4-Benzylamino-3-fluoro-piperidine-1-carboxylic acid tert-butyl ester

To a solution of the fluoride (40c) (7.12 g) in 1,2-dichloroethane (150 ml), benzylamine (4 ml) and subsequently sodium triacetoxy borohydride (8.5 g) were added and stirred overnight at room temperature. Thereafter, a saturated solution of sodium hydrogencarbonate (100 ml) was added, and the pH value was adjusted to 8 with solid sodium hydrogencarbonate. The phases were separated, and the aqueous phase is extracted with dichloromethane. The combined organic phases were washed with a saturated solution of sodium chloride, dried over sodium sulfate, filtered and concentrated. The residue was purified by flash chromatography (silica gel, hexane:acetic acid ethyl ester: 1:1), and resulted in the desired product.

MS (EI): m/z: 309 [M+H]⁺

### 40e) 4-Amino-3-fluoro-piperidine-1-carboxylic acid tert-butyl ester

To a solution of the benzylamine (40d) (4 g) in methanol (100 ml), 20% palladium hydroxide (2.7 g) was added and the reaction mixture was stirred under a hydrogen gas atmosphere for 4 hours. The solution was filtered, concentrated under vacuum and resulted in the desired product (2.84 g).

MS (EI): m/z: 219 [M+H]⁺

### 40f) 4-Benzyloxycarbonylamino-3-fluoro-piperidine-1-carboxylic acid tert-butyl ester

To a solution of the amine (40e) (2.84 g) in acetic acid ethyl ester (50 ml) and a saturated solution of sodium hydrogencarbonate (50 ml), Z-chloride (2 ml) was added and stirred for 1 hour at room temperature. The two phases were separated, and the aqueous phase was extracted with acetic acid ethyl ester. The combined organic phases were washed with a saturated solution of sodium chloride, dried over sodium sulfate, filtered and concentrated. The residue was purified by flash chromatography (silica gel, hexane:acetic acid ethyl ester: 2:1) and resulted in the desired product.

MS (EI): m/z: 353 [M+H]⁺

### 40g) (3-Fluoro-piperidin-4-yl)-carbamic acid benzyl ester

A solution of the protected amine (40f) in TFA (10 ml) was stirred at room temperature for 30 minutes. The solvent was removed under reduced pressure, and the residue was resuspended or redissolved, respectively, in a 3 N solution of sodium hydroxide and extracted several times with dichloromethane:methanol 9:1. The combined organic phases were washed with as saturated solution of sodium chloride, dried over sodium sulfate, filtered, concentrated and resulted in the desired product (2.9 g).

MS (EI): m/z: 253 [M+H]⁺

### 40h) (3-Fluoro-1-[2-hydroxy-2-(3-methoxy-quinolin-5-yl)-ethyl]-piperidin-4-yl}-carbamic acid benzyl ester (enantiomer 1)

To a solution of 3-methoxy-5-oxiranyl quinoline (7a) (0.8 g) and piperidine (40g) (1.01 g) in DMF (10 ml), lithium perchlorate (0.425 g) was added and heated to 80° C. overnight. The solvent was removed under reduced pressure, and the residue was purified by flash chromatography (silica gel, acetic acid ethyl ester) and resulted in the desired product (1.7 g).

MS (EI): m/z: 454 [M+H]⁺

### 40i) 2-(4-Amino-3-fluoro-piperidin-1-yl)-1-(3-methoxy-quinolin-5-yl)-ethanol (enantiomer 1)

To a solution of the compound (40h) (1.7 g) in acetic acid ethyl ester (50 ml) and ethanol (10 ml), 10% palladium on charcoal (0.7 g) was added and stirred for 6 hours under a hydrogen gas atmosphere. The solution was filtered, concentrated and resulted in the desired product.

MS (EI): m/z: 320 [M+H]⁺

### 40j) Title Compound

The compound was prepared as in example 7d starting from the amine (40i) and 2,3-dihydro-benzo[1,4]dioxine-6-carbaldehyde.

MS (EI): m/z: 468 [M+H]⁺

### Example 41

### 6-({3-Fluoro-1-[2-hydroxy-2-(3-methoxy-quinolin-5-yl)-ethyl]-piperidin-4-ylamino}-methyl)-4H-benzo[1,4]oxazin-3-one (enantiomer 1)

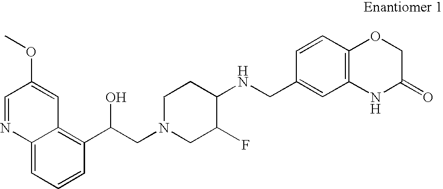

The compound was prepared as in example 40j starting from the aldehyde (1j).

MS (EI): m/z: 481 [M+H]⁺

### Example 42

### 2-[3-Fluoro-4-((E)-3-phenyl-allylamino)-piperidin-1-yl]-1-(3-methoxy-quinolin-5-yl)-ethanol (enantiomer 1)

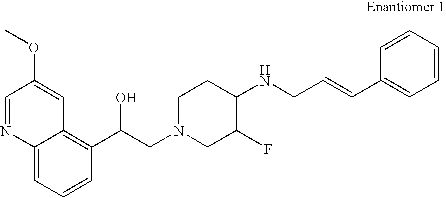

The compound was prepared as in example 40j starting from cinnamic aldehyde. MS (EI): m/z: 436 [M+H]⁺

### Example 43

### 6-({3-Fluoro-1-[2-hydroxy-2-(3-methoxy-quinolin-5-yl)-ethyl]-piperidin-4-ylamino}-methyl)-4H-pyrido[3,2-b][1,4]thiazin-3-one (enantiomer 1)

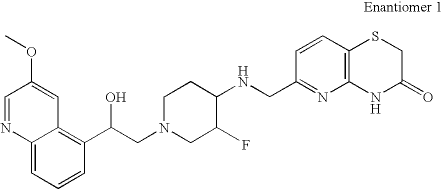

The compound was prepared as in example 40j starting from the aldehyde (2h).

MS (EI): m/z: 498 [M+H]⁺

### Example 44

### 2-{4-[(3,4-Dihydro-2H-benzo[1,4]oxazin-6-ylmethyl)-amino]-3-fluoro-piperidin-1-yl}-1-(3-methoxy-quinolin-5-yl)-ethanol (enantiomer 1)

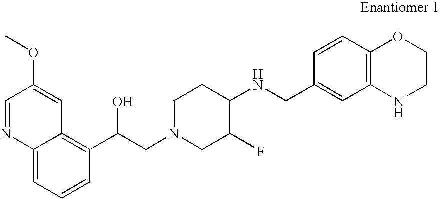

### 44a) (3,4-Dihydro-2H-benzo[1,4]oxazin-6-yl)-methanol

To an ice cold solution of the aldehyde (1j) (1.77 g) in THF (100 ml), lithium aluminium hydride (1 g) was added, stirred for 30 minutes at 0° C., and subsequently heated to reflux for 90 minutes. After cooling water (1 ml), a 15° A) solution of sodium hydroxide (1 ml) and water (3 ml) were added subsequently. The reaction mixture was diluted with THF (100 ml), and the precipitate was separated by filtration. The filtrate was concentrated and the residue was purified by flash chromatography (silica gel, acetic acid ethyl ester) and resulted in the desired product (1.5 g).

MS (EI): m/z: 166 [M+H]⁺

### 44b) 3,4-Dihydro-2H-benzo[1,4]oxazine-6-carbaldehyde

To a solution of the alcohol (44a) (1.5 g) in dichloromethane (100 ml) and THF (100 ml), manganese dioxide (3 g) was added. After stirring for 2 hours at room temperature, further manganese dioxide (3 g) was added thereto and stirred for further 3 hours. Thereafter, the reaction mixture was filtered through Celit and rinsed again with THF. The filtrate was concentrated and resulted in the desired product (1 g).

MS (EI): m/z: 164 [M+H]⁺

### 44c) Title Compound

The title compound was prepared as in example 40j starting from the aldehyde (44b).

MS (EI): m/z: 467 [M+H]⁺

### Example 45

### 2-{4-[(2,3-Dihydro-[1,4]dioxino[2,3-c]pyridin-7-ylmethyl)-1.0 amino]-3-fluoro-piperidin-1-yl}-1-(3-methoxy-quinolin-5-yl)-ethanol (enantiomer 1)

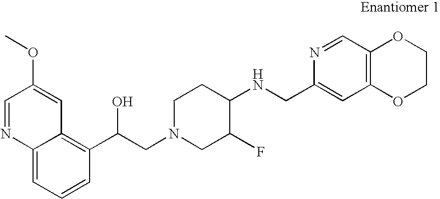

The compound was prepared as in example 40j starting from the aldehyde (5d).

MS (EI): m/z: 470 [M+H]⁺

### Example 46

### 2-{4-[(Benzo[1,2,5]thiadiazol-5-ylmethyl)-amino]-3-fluoro-piperidin-1-yl}-1-(3-methoxy-quinolin-5-yl)-ethanol (enantiomer 1)

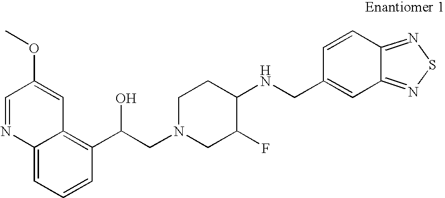

The compound was prepared as in example 40j starting from benzo[1,2,5]thiadiazole-5-carbaldehyde.

MS (EI): m/z: 468 [M+H]⁺

### Example 47

### 6-({3-Fluoro-1-[2-hydroxy-2-(3-methoxy-quinolin-5-yl)-ethyl]-piperidin-4-ylamino}-methyl)-4H-benzo[1,4]thiazin-3-one (enantiomer 1)

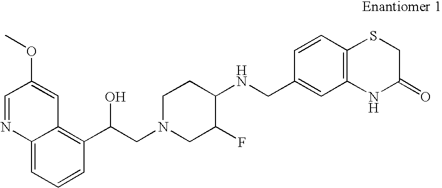

The compound was prepared as in example 40j starting from the aldehyde (4b).

MS (EI): m/z: 497 [M+H]⁺

### Example 48

### 2-[4-[(2,3-Dihydro-benzo[1,4]dioxin-6-ylmethyl)-amino]-4-(2-hydroxy-ethyl)-piperidin-1-yl]-1-(3-methoxy-quinolin-5-yl)-ethanol (racemate)

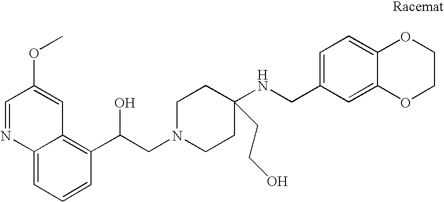

### 48a) 4-Amino-4-carboxymethyl-piperidine-1-carboxylic acid benzyl ester

To a solution of 4-oxo-piperidine-1-carboxylic acid benzyl ester (10 g) and ammonium formiate (4.93 g) in methanol (20 ml), malonic acid (4.5 g) was added and heated to reflux for 3 days. After concentrating, the crude product (12 g) was processed further without any additional purification.

### 48b) 4-Amino-4-methoxycarbonylmethyl-piperidine-1-carboxylic acid benzyl ester

To a solution of the ester (48a) (5 g) in methanol (25 ml) and hexane (25 ml), TMS-diazomethane (2 M in hexane, 9 ml) was added thereto and stirred for 3 hours at room temperature. After concentrating, the residue was resuspended or redissolved, respectively, in acetic acid ethyl ester (100 ml) and 1 N solution of sodium hydroxide (30 ml). The organic phase was washed with a 1 N solution of sodium hydroxide (30 ml) and a saturated solution of sodium chloride (30 ml), dried over sodium sulfate, filtered and concentrated, and resulted in the desired product (4.9 g).

MS (EI): m/z: 307 [M+H]⁺

### 48c) 4-[(2,3-Dihydro-benzo[1,4]dioxin-6-ylmethyl)-amino]-4-methoxycarbonylmethyl-piperidine-1-carboxylic acid benzyl ester

To a solution of the aminopiperidine (48b) (0.86 g) and 2,3-dihydro-benzo[1,4]-dioxine-6-carbaldehyde (0.5 g) in 1,2-dichloroethane (15 ml), sodium triacetoxy borohydride (0.72 g) was added and stirred for 16 hours at room temperature. Thereafter, a saturated solution of sodium bicarbonate (20 ml) and dichloromethane (50 ml) were added, the phases were separated and the aqueous phase was extracted with dichloromethane (50 ml). The combined organic phases were washed with a saturated solution of sodium chloride, dried over sodium sulfate, filtered and concentrated. The residue was purified by flash chromatography (silica gel, acetic acid ethyl ester) and resulted in the desired product (0.75 g).

MS (EI): m/z: 455 [M+H]⁺

### 48d) 4-[(2,3-Dihydro-benzo[1,4]dioxin-6-ylmethyl)-amino]-4-(2-hydroxyethyl)-piperidine-1-carboxylic acid benzyl ester

To a solution of the ester (48c) (0.75 g) in THF (20 ml), lithium borohydride (0.3 g) was added and stirred for 2 hours at room temperature. Water (5 ml), methanol (2 ml) and a saturated solution of potassium sodium tartrate (50 ml) were added. After stirring for 20 minutes, dichloromethane (100 ml) was added, the phases were separated and the aqueous phase is extracted with dichloromethane (3×50 ml). The combined organic phases were concentrated, and the residue is purified by flash chromatography (silica gel, acetic acid ethyl ester) and resulted in the desired product (0.5 g).

MS (EI): m/z: 427 [M+H]⁺

### 48e) 2-{4-[(2,3-Dihydro-benzo[1,4]dioxin-6-ylmethyl)-amino]-piperidin-4-yl}-ethanol

To a solution of the protected piperidine (48d) (0.5 g) in THF (8 ml) and methanol (2 ml), 20% palladium hydroxide (0.5 g) was added and stirred for 4 hours at room temperature under a hydrogen gas atmosphere. The solution was filtered, concentrated and resulted in the desired product (340 mg).

MS (EI): m/z: 293 [M+H]⁺

### 48f) Title Compound

The title compound was prepared as in example 1g starting from the epoxide (14b) and the piperidine (48e).

MS (EI): m/z: 494 [M+H]⁺

### Example 49

### 2-[4-[(Benzo[1,3]dioxol-5-ylmethyl)-amino]-4-(2-hydroxyethyl)-piperidin-1-yl]-1-(3-methoxy-quinolin-5-yl)-ethanol (racemate)

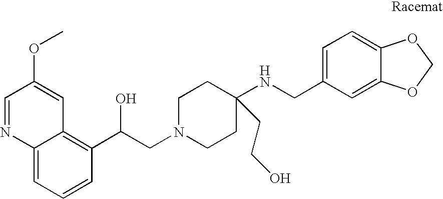

The piperidine (2-{4-[benzo[1,3]dioxol-5-ylmethyl)-amino]-piperidin-4-yl}-ethanol) was prepared analogically to the steps 48c to 48e starting from benzo[1,3]dioxol-5-carbaldehyde. The title compound was prepared as in example 48f starting from the epoxide (14b) and 2-{4-[(benzo[1,3]dioxol-5-ylmethyl)-amino]-piperidin-4-yl}-ethanol.

MS (EI): m/z: 480 [M+H]⁺

### Example 50

### 6-({4-(2-Hydroxy-ethyl)-1-[2-hydroxy-2-(3-methoxy-quinolin-5-yl)-ethyl]-piperidin-4-ylamino}-methyl)-4H-benzo[1,4]oxazin-3-one (racemate)

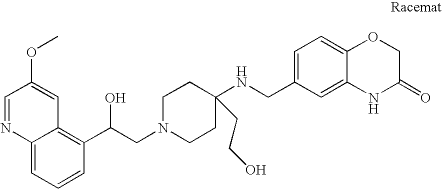

The piperidine (6-{[4-(2-hydroxy-ethyl)-piperidin-4-ylamino]-methyl}-4H-benzo[1,4]oxazin-3-one) was prepared analogically to the steps 48c to 48e starting from the aldehyde (1j). The title compound was prepared as in example 48f starting from the epoxide (14b) and 6-{[4-(2-hydroxy-ethyl)-piperidin-4-ylamino]-methyl}-4H-benzo[1,4]oxazin-3-one.

MS (EI): m/z: 507 [M+H]⁺

### Example 51

### 2-{4-[(2,3-Dihydro-benzo[1,4]dioxin-6-ylmethyl)-amino]-3-hydroxymethyl-piperidin-1-yl}-1-(3-methoxy-quinolin-5-yl)-ethanol (racemate)

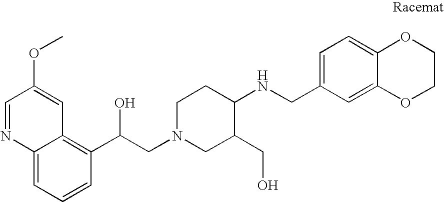

### 51a) 1-Benzyl-3-hydroxymethyl-piperidin-4-ol

To a mixture of sodium hydroxide (1.344 g) and 1-benzyl-3-carbethoxy-4-piperidone hydrochloride (10 g) in methanol (160 ml), sodium borohydride (2.543 g) was added at 0° C. and stirred for 30 minutes. Thereafter, water (150 ml) was added in drops, and the solution was partially concentrated. The aqueous phase was extracted with dichloromethane (3×150 ml). The combined organic phases were washed with a saturated solution of sodium chloride, dried over sodium sulfate, filtered and concentrated. The residue was resuspended or redissolved, respectively, in ether (140 ml), and lithium aluminum hydride (2.55 g) was added at 0° C. and stirred for 1 hour. Thereafter, water (2 ml), a 3 N solution of sodium hydroxide (4 ml) and water (9 ml) were added subsequently, heated to room temperature and ether (150 ml) was added. The solid was removed by filtration; the filtrate was concentrated and resulted in the desired product (5.33 g).

MS (EI): m/z: 222 [M+H]⁺

### 51b) 1-Benzyl-3-(tert-butyl-dimethyl-silanyloxymethyl)-piperidin-4-ol

To a solution of the diol (51a) (5.33 g) in dichloromethane (47 ml), tert-butyldimethylsilyl chloride (3.9 g), triethylamine (6.6 ml) and DMAP (0.287 mg) were added at 0° C. and stirred for 4 days at 0° C. After concentrating, the residue was resuspended or redissolved, respectively, in water and acetic acid ethyl ester, the phases were separated and the aqueous phase was extracted with acetic acid ethyl ester (3×100 ml). The combined organic phases were washed with a saturated solution of sodium chloride, dried over sodium sulfate, filtered and concentrated. The residue was purified by flash chromatography (silica gel, acetic acid ethyl ester:hexane: 1:2) and resulted in the desired product (5.6 g).

MS (EI): m/z: 336 [M+H]⁺

### 51c) 3-(tert-Butyl-dimethyl-silanyloxymethyl)-piperidin-4-ol

To a solution of benzylpiperidine (51b) (5.6 g) in THF:methanol 1:1 (90 ml), 10% palladium hydroxide (3.6 g) were added and stirred overnight in a hydrogen gas atmosphere. Then, the reaction mixture was filtered and the filtrate was concentrated and resulted in the desired product (4.1 g).

MS (EI): m/z: 246 [M+H]⁺

### 51d) 3-(tert-Butyl-dimethyl-silanyloxymethyl)-4-hydroxy-piperidine-1-carboxyl acid benzyl ester

To a solution of the piperidinol (51c) (4.1 g) in acetone:water 2:1 (87 ml), sodium bicarbonate (2.803 g) and Z-chloride (2.35 ml) were added. After stirring for 1 hour at room temperature, the solution was partially concentrated, and the aqueous phase was extracted with acetic acid ethyl ester. The combined organic phases were washed with a saturated solution of sodium chloride, dried over magnesium sulfate, filtered and concentrated. The residue was purified by flash chromatography (silica gel, acetic acid ethyl ester:hexane: 1:4, 1:1), and resulted in the desired product (6.1 g).

MS (EI): m/z: 380 [M+H]⁺

### 51e) 3-(tert-Butyl-dimethyl-silanyloxymethyl)-4-oxo-piperidine-1-carboxylic acid benzyl ester

To a solution of the alcohol (51d) (3 g), N-methylmorpholine N-oxide (1.857 g) and powdered molecular sieve 4A (3.95 g) in dichloromethane (15 ml), TPAP (0.139 g) was added at room temperature. After one hour, the solution was filtered through silica gel, and the filtrate was concentrated. The residue was purified by flash chromatography (silica gel, acetic acid ethyl ester:hexane 1:4) and resulted in the desired product (2 g).

MS (EI): m/z: 378 [M+H]⁺

### 51f-Amino-3-(tert-butyl-dimethyl-silanyloxymethyl)-piperidine-1-carboxylic acid benzyl ester

To a solution of the ketone (51e) (2 g) in methanol (50 ml), ammonium acetate (6.13 g) and sodium triacetoxy borohydride (1.69 g) were added and stirred overnight at room temperature. After concentrating the reaction mixture, the residue was resuspended or redissolved, respectively, in water and dichloromethane, the phases were separated and the aqueous phase was extracted with dichloromethane (3×70 ml). The combined organic phases were washed with a saturated solution of sodium chloride, dried over sodium sulfate, filtered and concentrated. The crude product (1 g) was used further without any additional purification.

### 51g) 3-(tert-Butyl-dimethyl-silanyloxymethyl)-4-[(2,3-dihydro-benzo[1,4]-dioxin-6-ylmethyl)-amino]-piperidine-1-carboxylic acid benzyl ester

To a solution of the amine (51f) (1 g) in methanol (7 ml) and dichloromethane (22 ml), molecular sieve 3A (7 g) and 2,3-dihydro-benzo[1,4]dioxine-6-carbaldehyde (0.434 g) were added and stirred for 20 hours at room temperature. Then, sodium borohydride (0.120 g) was added and stirred for further 2 hours. The molecular sieve was separated by filtration, and the filtrate was washed with a saturated solution of sodium bicarbonate and a saturated solution of sodium chloride. The organic phase was dried over magnesium sulfate, filtered and concentrated. The residue was purified by flash chromatography (silica gel, dichloromethane:methanol: 9:1+1% ammonia) and resulted in the desired product (500 mg).

MS (EI): m/z: 527 [M+H]⁺

### 51h) [3-(tert-Butyl-dimethyl-silanyloxymethyl)-piperidin-4-yl]-(2,3-dihydro-benzo[1,4]dioxin-6-ylmethyl)-amine

To a solution of the Z-protected piperidine (51g) (500 mg) in acetic acid ethyl ester (15 ml), 10% palladium on charcoal (0.4 g) was added and stirred for 12 hours under a hydrogen gas atmosphere. The solution was filtered and concentrated, and resulted in the desired product (0.37 g).

MS (EI): m/z: 393 [M+H]⁺

### 51i) 2-{3-(tert-Butyl-dimethyl-silanyloxymethyl)-4-[(2,3-dihydro-benzo-[1,4]dioxin-6-ylmethyl)-amino]-piperidin-1-yl}-1-(3-methoxy-quinolin-5-yl)-ethanol

To a solution of the epoxide (14b) (150 mg) and the protected piperidine (51h) (293 mg) in DMF (3 ml), potassium carbonate (0.150 g) and lithium perchlorate (0.083 g) were added and stirred overnight at 80° C. After cooling, the solution was filtered and concentrated. The residue was purified by flash chromatography (silica gel, dichloromethane:methanol: 9:1) and resulted in the desired product (304 mg).

MS (EI): m/z: 594 [M+H]⁺

### 51j) Title Compound

To a solution of the silylether (51i) (304 mg) in acetonitrile (1 ml), a 2.5 N aqueous solution of hydrofluoric acid (0.62 ml) was added at 0° C. and stirred for 1 hour. Thereafter, the reaction mixture is made alkaline with a 3 N solution of sodium hydroxide (1 ml) and the solution is concentrated. The residue was purified by flash chromatography (silica gel, dichloromethane:methanol: 9:1+1% ammonia) and resulted in the desired product (112 mg).

MS (EI): m/z: 480 [M+H]⁺

### Example 52

### 6-({1-[2-Hydroxy-2-(3-methoxy-quinolin-5-yl)-ethyl]-3-hydroxymethyl-piperidin-4-ylamino}-methyl)-4H-benzo[1,4]oxazin-3-one (racemate)

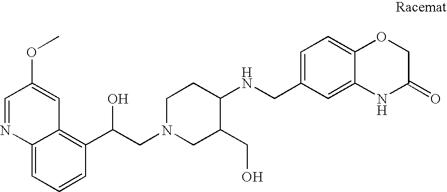

The piperidine (6-{[3-(tert-butyl-dimethyl-silanyloxy-methyl)-piperidin-4-ylamino]-methyl}-4H-benzo[1,4]oxazin-3-one) was prepared analogically to the steps 51g to 51h starting from the aldehyde (1j). The title compound was prepared as in example 51i to 51j from the epoxide (14b) and 6-{[3-(tert-butyl-dimethyl-silanyloxy-methyl)piperidin-4-ylamino]-methyl}-4H-benzo[1,4]oxazin-3-one.

MS (EI): m/z: 493 [M+H]⁺

### Example 53

### 1-(3-Methoxy-quinolin-5-yl)-2-{4-[3-(thiophen-2-ylsulfanyl)propyl]-piperazin-1-yl}-ethanol

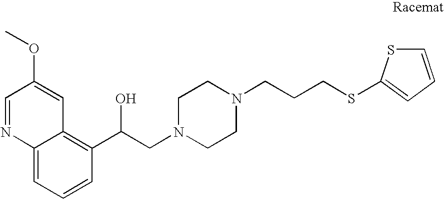

### 53a) 2-(3-Bromo-propylsulfanyl)-thiophene

To a solution of thiophene thiol (2.5 g) and sodium hydroxide (2 g) in water (10 ml), 1,3-dibromopropane (6.59 ml) was added and heated overnight to 80° C. After cooling, the reaction mixture was diluted with ether, the phases were separated and the aqueous phase was extracted with ether. The combined organic phases were washed with a saturated solution of sodium chloride, dried over sodium sulfate, filtered and concentrated. The residue was purified by flash chromatography (silica gel, hexane) and resulted in the desired product (2.37 g).

MS (EI): m/z: 238 [M+H]⁺

### 53b) 4-[3-(Thiophen-2-ylsulfanyl)-propyl]-piperazine-1-carboxylic acid tert-butyl ester

To a solution of piperazine-1-carboxylic acid tert-butyl ester (0.43 g) and the bromide

(53a) (0.5 g) in DMF (5 ml), potassium carbonate (0.4 g) was added and heated for 14 hours to 60° C. Then, the solution was concentrated and the residue was purified by flash chromatography (silica gel, dichloromethane:methanol: 9:1) and resulted in the desired product (500 mg).

MS (EI): m/z: 343 [M+H]⁺

### 53c) 1-[3-(Thiophen-2-ylsulfanyl)-propyl]-piperazine

A solution of the protected piperazine (53b) (0.5 g) in TFA (5 ml) was stirred for 20 minutes at room temperature. After concentrating the reaction mixture, the residue was resuspended or redissolved, respectively, in an 1 N solution of sodium hydroxide (30 ml) and extracted with dichloromethane (3×30 ml). The combined organic phases were dried over sodium sulfate, filtered and concentrated, and resulted in the desired product (346 mg).

MS (EI): m/z: 243 [M+H]⁺

### 53d) Title Compound

To a solution of the epoxide (14b) (0.1 g) and the piperazine (53c) (0.130 g) in DMF (5 ml), lithium perchlorate (0.06 g) and potassium carbonate (0.1 g) were added and heated for 2 hours to 40° C. Then, the solution was concentrated and the residue was purified by flash chromatography (silica gel, dichloromethane:methanol: 19:1) and resulted in the desired product.

MS (EI): m/z: 444 [M+H]⁺

### Example 54

### 6-(1-Hydroxy-2-{4-[2-hydroxy-2-(3-methoxy-quinolin-5-yl)ethyl]-piperazin-1-yl}-ethyl)-4H-benzo[1,4]oxazin-3-one (racemate)

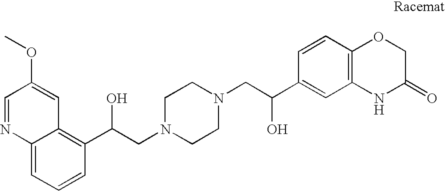

### 54a) 4-[2-Hydroxy-2-(3-oxo-3,4-dihydro-2H-benzo[1,4]oxazin-6-yl)-ethyl]-piperazine-1-carboxylic acid benzyl ester

To a solution of piperazine-1-carboxylic acid benzyl ester (2.2 g) in ethanol (20 ml) and acetonitrile (10 ml), 6-(2-chloro-acetyl)-4H-benzo[1,4]oxazin-3-one (2.25 g) and triethylamine (1.67 ml) were added and heated for 3 hours to 65° C. Then, the solution was concentrated, and the residue was resuspended or redissolved, respectively, in methanol (30 ml). After cooling to 0° C., sodium borohydride (2 g) was added in portions and stirred for 30 minutes. Then, water (20 ml) was added, and the solution was partially concentrated. The residue was extracted with acetic acid ethyl ester (3×100 ml). The combined organic phases were washed with a saturated solution of sodium chloride, dried over sodium sulfate, filtered and concentrated. The residue was purified by flash chromatography (silica gel, dichloromethane:methanol: 19:1) and resulted in the desired product (2 g).

MS (EI): m/z: 412 [M+H]⁺

### 54b) 6-(1-Hydroxy-2-piperazin-1-yl-ethyl)-4H-benzo[1,4]oxazin-3-one

To a solution of the protected piperazine (54a) (3 g) in acetic acid ethyl ester (100 ml), 10% palladium on charcoal (1.5 g) was added. The reaction mixture was stirred overnight under a hydrogen gas atmosphere, and then filtered and concentrated and resulted in the desired product (2 g).

MS (EI): m/z: 278 [M+H]⁺

### 54c) Title Compound

The title compound was prepared as in example 53d starting from the epoxide (14b) and the piperazine (54b).

MS (EI): m/z: 479 [M+H]⁺

### Example 55

### 6-(1-Hydroxy-2-{4-[2-hydroxy-2-(3-methoxy-quinolin-5-yl)ethyl]-piperazin-1-yl}-ethyl)-4H-benzo[1,4]thiazin-3-one (racemate)

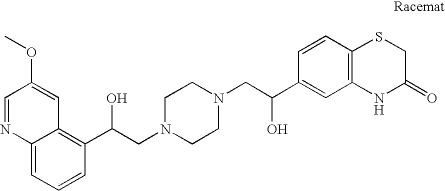

The piperazine (6-(1-hydroxy-2-piperazin-1-yl-ethyl)-4H-benzo[1,4]thiazin-3-one) was prepared analogically to the steps 54a to 54b starting from 6-(2-chloro-acetyl)-4H-benzo[1,4]thiazin-3-one. The title compound was prepared as in example 54c starting from the epoxide (14b) and 6-(1-hydroxy-2-piperazin-1-yl-ethyl)-4H-benzo[1,4]thiazin-3-one.

MS (EI): m/z: 495 [M÷H]⁺

### Example 56

### 2-{4-[2-(2,3-Dihydro-benzo[1,4]dioxin-6-yl)-2-hydroxy-ethyl]-piperazin-1-yl}-1-(3-methoxy-quinolin-5-yl)-ethanol (enantiomer 1)

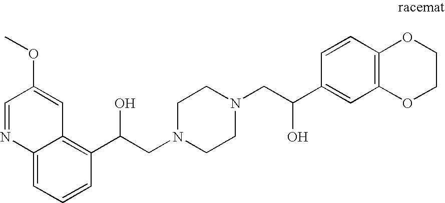

The piperazine (1-(2,3-dihydro-benzo[1,4]dioxin-6-yl)-2-piperazin-1-yl-ethanol) was prepared analogically to the steps 54a bis 54b starting from 2-chloro-1-(2,3-dihydrobenzo[1,4]dioxin-6-yl)-ethanone. The title compound was prepared as in example 54c starting from the epoxide (14b) and 1-(2,3-dihydro-benzo[1,4]dioxin-6-yl)-2-piperazin-1-yl-ethanol.

MS (EI): m/z: 466 [M+H]⁺

### Example 57

### 5-{2-[4-(2,3-Dihydro-benzo[1,4]dioxin-6-ylmethyl)-piperazin-1-yl]-ethoxy}-3-methoxy-quinoline

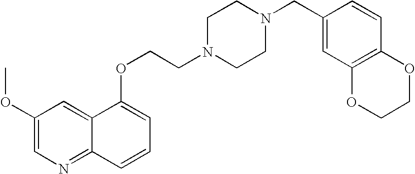

### 57a) 3-Methoxy-5-(4,4,5,5-tetramethyl[1,3,2]dioxaborolan-2-yl)-quinoline

To a mixture of bis(pinacolato)diborone (6.14 g), 1,1′-bis(diphenylphosphino) ferrocene palladium(II) dichloride dichloromethane complex (1.475 g) and potassium acetate (5.93 g), a solution of 1b (4.8 g) in DMSO (145 ml) was added. The reaction mixture was stirred overnight at 80° C. After cooling to room temperature, the reaction mixture was diluted with water (300 ml) and acetic acid ethyl ester (300 ml). The phases were separated, and the aqueous phase was extracted with acetic acid ethyl ester (2×300 ml). The combined organic phases were washed with a saturated solution of sodium chloride, dried over sodium sulfate, filtered and concentrated. The brown residue was purified by flash chromatography (silica gel, acetic acid ethyl ester:hexane: 1:4) and resulted in the desired product (4.65 g).

MS (EI): m/z: 286 [M+H]⁺

### 57b) 3-Methoxy-quinolin-5-ol

To an ice cold solution of 57a (4.6 g) in THF (110 ml), a 3 N solution of sodium hydroxide (13 ml) was added in drops. Subsequently, a 30% aqueous solution of hydrogen peroxide (5.6 ml) was added in drops, and the reaction mixture was stirred for one hour at 0° C. The reaction mixture was resuspended or redissolved, respectively, in water (100 ml) and once extracted with acetic acid ethyl ester (200 ml). The pH value of the aqueous phase was adjusted to 4 with a 1 N solution of hydrochloric acid, and subsequently, the reaction mixture was extracted with acetic acid ethyl ester (3×100 ml). The organic phases were combined, dried over sodium sulfate, filtered and concentrated. The residue was purified by flash chromatography (silica gel, dichloromethane:methanol: 29:1) and resulted in the desired product (2.82 g).

MS (EI): m/z: 176 [M+H]⁺

### 57c) 4-(2-Hydroxy-ethyl)-piperazine-1-carboxylic acid benzyl ester

To a solution of hydroxyethyl piperazine (13 ml) in acetone (200 ml), a 10% solution of sodium hydrogencarbonate (254 ml) was added under vigorous stirring. Subsequently, the reaction mixture was cooled to 0° C. and benzyl chloroformate (17.92 ml) was added in drops. The reaction mixture was stirred for 4 hours at room temperature. After removing the acetone under vacuum, the aqueous phase was extracted with acetic acid ethyl ester (3×250 ml). The combined organic phases were washed once with a saturated solution of sodium chloride, dried over sodium sulfate, filtered, concentrated and resulted in the desired product (28.2 g).

MS (EI): m/z: 265 [M+H]⁺

### 57d) 4-(2-Methanesulfonyloxy-ethyl)-piperazine-1-carboxylic acid benzyl ester

A solution of 57c (2.0 g) in dichloromethane (10 ml) was cooled to 0° C. and mixed with triethylamine (1.27 ml) and methane sulfonylchloride (706 μl). The solution was thawed up to room temperature and then stirred for 30 minutes. The reaction mixture was diluted with dichloromethane and then washed subsequently with a saturated solution of sodium hydrogencarbonate, water and a saturated solution of sodium chloride. By concentrating the desired product was obtained (2.57 g).

MS (EI): m/z: 343 [M+H]⁺

### 57e) 4-[2-(3-Methoxy-quinolin-5-yloxy)-ethyl]-piperazine-1-carboxylic acid benzyl ester

The compound (57b) (350 mg) was dissolved in DMF (2 ml) and mixed at room temperature with sodium hydride (87 mg). After stirring for 10 minutes, a solution of the compound (57d) (684 mg) in DMF (2 ml) was added slowly and in drops. The reaction mixture was stirred overnight at room temperature, and then resuspended or redissolved, respectively, in water and extracted with acetic acid ethyl ester (3×5 ml). The combined organic phases were washed several times with water, dried over sodium sulfate, filtered, concentrated, and resulted in the desired product (320 mg).

MS (EI): m/z: 422 [M+H]⁺

### 57f) 3-Methoxy-5-(2-piperazin-1-yl-ethoxy)-quinoline

To a solution of the compound (57e) (250 mg) in acetic acid ethyl ester (25 ml) and methanol (25 ml), 10% palladium on charcoal (50 mg) was added and stirred for 4 hours under a hydrogen gas atmosphere. The solution was filtered, concentrated and resulted in the desired product.

MS (EI): m/z: 288 [M+H]⁺

### 57g) Title Compound

The title compound was prepared as in example 1k starting from 3-methoxy-5-(2-piperazin-1-yl-ethoxy)-quinoline (57f) and 2,3-dihydro-benzo[1,4]dioxine-6-carbaldehyde in a yield of 66%.

MS (EI): m/z: 436 [M+1-1]⁺

### Example 58

### 6-{4-[2-(3-Methoxy-quinolin-5-yloxy)-ethyl]-piperazin-1-yl-methyl}-4H-benzo[1,4]oxazin-3-one

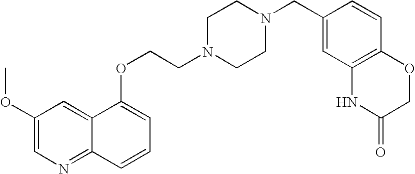

The title compound was prepared as in example 57g starting from the aldehyde (1j) in a yield of 71%.

MS (EI): m/z: 449 [M+H]⁺

### Example 59

### 6-{4-[2-(3-Methoxy-quinolin-5-yloxy)-ethyl]-piperazin-1-ylmethyl}-4H-benzo[1,4]thiazin-3-one

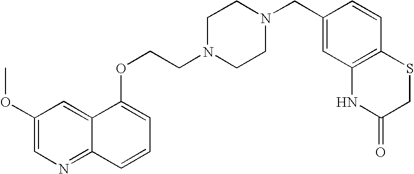

The title compound was prepared as in example 57g starting from the aldehyde (4b) in a yield of 56%.

MS (EI): m/z: 465 [M+H]⁺

### Example 60

### 6-{4-[2-(3-Methoxy-quinolin-5-yloxy)-ethyl]-piperazin-1-ylmethyl}-4H-pyrido[3,2-b][1,4]thiazin-3-one

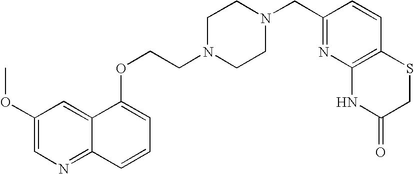

The title compound was prepared as in example 57g starting from the aldehyde (2h) in a yield 80%.

MS (EI): m/z: 466 [M+H]⁺

### Example 61

### 5-(2-{4-[(E)-3-(2,5-Difluorophenyl)-allyl]-piperazin-1-yl}-ethoxy)-3-methoxy-quinoline

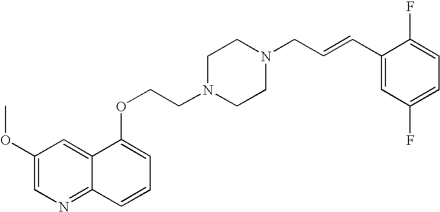

The title compound was prepared as in example 57g starting from (E)-3-(2,5-difluorophenyl)-propenal (this compound was prepared according to WO 2004/087 647).

MS (EI): m/z: 440 [M+H]⁺

### Example 62

### 3-Methoxy-5-[2-(4-naphthalen-2-ylmethyl-piperazin-1-yl)-ethoxy]-quinoline

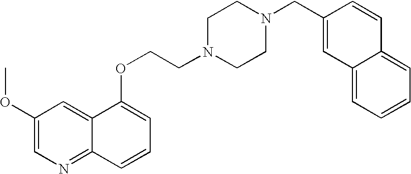

The title compound was prepared as in example 57g starting from naphthalene 2-carbaldehyde.

MS (EI): m/z: 428 [M+H]⁺

### Example 63

### 3-Methoxy-5-(2-{4-[2-(thiophen-2-yl-sulfanyl)-ethyl]-piperazin-1-yl}-ethoxy)-quinoline

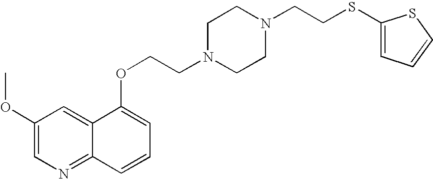

63a) 2-(2-Bromo-ethylsulfanyl)-thiophene

The compound was prepared as in example 53a starting from 1,2-dibromoethane.

MS (EI): m/z: 224 [M+H]⁺

### 63b) Title Compound

The compound was prepared as in example 53b starting from 3-methoxy-5-(2-piperazin-1-yl-ethoxy)-quinoline (57f) and 2-(2-bromo-ethylsulfanyl)-thiophene (63a) in a yield of 86%.

MS (EI): m/z: 430 [M+H]⁺

### Example 64

### 6-{4-[2-(3-Methoxy-quinolin-5-yloxy)-ethyl]-piperazine-1-carbonyl}-4H-benzo[1,4]thiazin-3-one

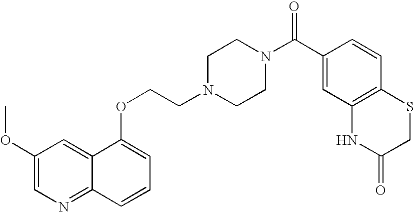

The title compound was prepared as in example 33 starting from 3-methoxy-5-(2-piperazin-1-yl-ethoxy)-quinoline (57f) and 3-oxo-3,4-dihydro-2H-benzo[1,4]thiazine-6-carboxylic acid.

MS (EI): m/z: 479 [M+H]⁺

### Example 65

### 6-{4-[2-(3-Methoxy-quinolin-5-yloxy)-ethyl]-piperazine-1-carbonyl}-4H-benzo[1,4]oxazin-3-one

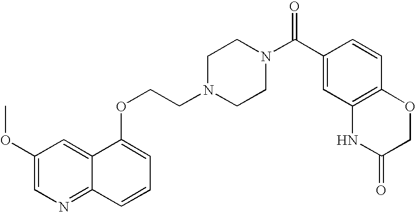

The title compound was prepared as in example 33 starting from 3-methoxy-5-(2-piperazin-1-yl-ethoxy)-quinoline (57f) and 3-oxo-3,4-dihydro-2H-benzo[1,4]oxazine-6-carboxylic acid.

MS (EI): m/z: 463 [M+H]⁺

### Example 66

### 6-{4-[2-(3-Methoxy-quinolin-5-yloxy)-ethyl]piperazine-1-sulfonyl}-4H-benzo[1,4]thiazin-3-one

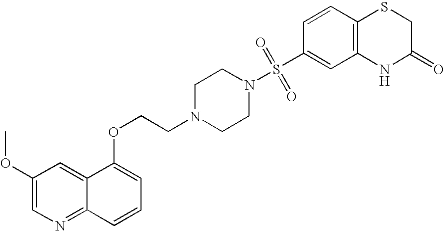

The compound was prepared as in example 37 starting from 3-methoxy-5-(2-piperazin-1-yl-ethoxy)-quinoline (57f) and 3-oxo-3,4-dihydro-2H-benzo[1,4]thiazine-6-sulfonylchloride.

MS (EI): m/z: 515 [M+H]⁺

### Example 67

### 6-{4-[2-(3-Methoxy-quinolin-5-yloxy)-ethyl]piperazine-1-sulfonyl}-4H-benzo[1,4]oxazin-3-one

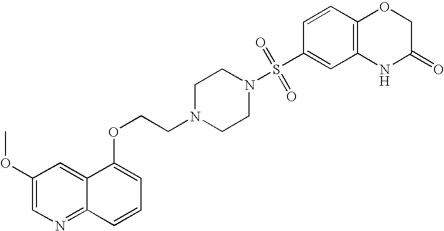

The title compound was prepared as in example 37 starting from 3-methoxy-5-(2-piperazin-1-yl-ethoxy)-quinoline (57f) and 3-oxo-3,4-dihydro-2H-benzo[1,4]oxazine-6-sulfonylchloride.

MS (EI): m/z: 499 [M+H]⁺

### Example 68

### 2-{4-[2-(2,3-Dihydro-benzo[1,4]dioxin-6-yl)-ethyl]-piperazin-1-yl}-1-(3-methoxy-quinolin-5-yl)-ethanol (enantiomer 1)

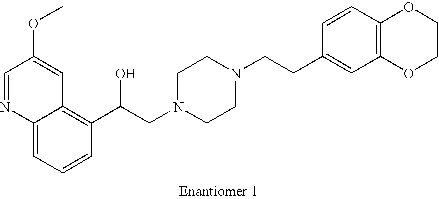

### 68a) 6-Vinyl-2,3-dihydro-benzo[1,4]dioxine

A solution of methyltriphenyl phosphonium bromide (13.2 g) in THF (120 ml) was cooled to −78° C., and then, butyl lithium (15 ml, 2.5 M solution in hexane) was added, and subsequently stirred for 15 minutes at −78° C., and then stirred further for 45 minutes at 0° C. After cooling to −78° C., a solution of 2,3-dihydro-benzo[1,4]dioxine-6-carbaldehyde (5 g) in THF (20 ml) was added, heated to room temperature and stirred for 2 hours. The reaction mixture was washed with acetic acid ethyl ester (3×100 ml) and the combined organic phases were washed with a saturated solution of sodium chloride, dried over sodium sulfate, filtered and concentrated. The residue was purified by flash chromatography (silica gel, acetic acid ethyl ester:hexane: 1:6) and resulted in the desired product (3.9 g).

MS (EI): m/z: 163 [M+H]⁺

### 68b) 2-(2,3-Dihydro-benzo[1,4]dioxin-6-yl)-ethanol

To a solution of the vinyl compound (68a) (5 g) in THF (100 ml), 9-BBN (2.2 g) was added and stirred for 16 hours at room temperature. Thereafter, the reaction mixture was cooled to 0° C., and ethanol (20 ml), a 3 N solution of sodium hydroxide (110 ml) and a 30% solution of hydrogen peroxide (110 ml) were added. The reaction mixture was stirred for 1 hour at room temperature, and then mixed with a 10% solution of sodium sulfite (120 ml) and stirred for further 30 minutes. The phases were separated and the aqueous phase was extracted with acetic acid ethyl ester (2×100 ml). The combined organic phases were washed with a saturated solution of sodium chloride, dried over sodium sulfate, filtered and concentrated. The residue was purified by flash chromatography (silica gel, acetic acid ethyl ester:hexane: 2:1) and resulted in the desired product (3.75 g).

MS (EI): m/z: 181 [M+H]⁺

### 68c) Toluene-4-sulfonic acid 2-(2,3-dihydro-benzo[1,4]dioxin-6-yl)-ethyl ester

To a solution of the alcohol (68b) (3.55 g) in dichloromethane (70 ml), DMAP (4.2 g) and tosyl chloride (4.13 g) were added at 0° C. The solution was stirred for 20 minutes at 0° C. and then heated to room temperature. After 2 hours, the solution was concentrated, and the residue was purified by flash chromatography (silica gel, acetic acid ethyl ester:hexane: 1:3), and resulted in the desired product (3.23 g).

MS (EI): m/z: 335 [M+H]⁺

### 68d) 4-[2-(2,3-Dihydro-benzo[1,4]dioxin-6-yl)-ethyl]-piperazine-1-carboxylic acid benzyl ester

To a solution of the tosylate (68c) (0.7 g) in DMF (10 ml), piperazine-1-carboxylic acid benzyl ester (0.49 g) and triethylamine (1 ml) were added. The reaction mixture was heated for 16 hours to 60° C. and then concentrated. The residue was purified by flash chromatography (silica gel, acetic acid ethyl ester:hexane: 2:1) and resulted in the desired product (0.6 g).

MS (EI): m/z: 383 [M+H]⁺

### 68e) 1-[2-(2,3-Dihydro-benzo[1,4]dioxin-6-yl)-ethyl]-piperazine

To a solution of the protected piperazine (68d) (0.180 g) in acetic acid ethyl ester (5 ml), 10% palladium on charcoal (0.18 g) was added and hydrogenated at room temperature for 1 hour under a hydrogen gas atmosphere. The reaction mixture was filtered, the filtrate was concentrated and resulted in the desired product (0.13 g).

MS (EI): m/z: 249 [M+H]⁺

### 68f) Title Compound

To a solution of the epoxide (7a, enantiomer 1) (0.1 g) and the piperazine (68e) (0.13 g) in DMF (2 ml), lithium perchlorate (0.06 g) was added and the reaction mixture was heated for 4 hours to 60° C. Then, the solution was concentrated and the residue was purified by flash chromatography (silica gel, dichloromethane:methanol: 19:1) and resulted in the desired product.

MS (EI): m/z: 450 [M+H]⁺

### Example 69

### 6-[2-{4-(2-Hydroxy-2-(3-methoxy-quinolin-5-yl)-ethyl]-piperazin-1-yl}-ethyl)-4H-benzo[1,4]oxazin-3-one (enantiomer 1)

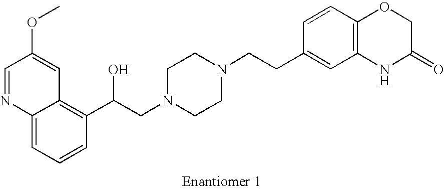

The piperazine (6-(2-piperazin-1-yl-ethyl)-4H-benzo[1,4]oxazin-3-one) was prepared analogically to the reaction steps 68a to 68e starting from the aldehyde (1j). The title compound was prepared as in example 68f starting from the epoxide (7a) and 1-(2,3-dihydro-benzo[1,4]dioxin-6-yl)-2-piperazin-1-yl-ethanol.

MS (EI): m/z: 463 [M+H]⁺

### Example 70

### 2-[4-(2-Benzo[1,3]dioxol-5-yl-ethyl)-piperazin-1-yl]-1-(3-methoxy-quinolin-5-yl)-ethanol (enantiomer 1)

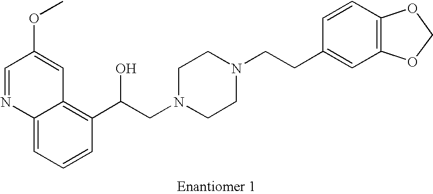

The piperazine (1-(2-benzo[1,3]dioxol-5-yl-ethyl)-piperazine) was prepared analogically to the reaction steps 68a to 68e starting from benzo[1,3]dioxol-5-carbaldehyde. The title compound was prepared as in example 68f starting from the epoxide (7a) and 1-(2-benzo[1,3]dioxol-5-yl-ethyl)-piperazine.

MS (EI): m/z: 436 [M+H]⁺

### Example 71

### 1-Cyclopropyl-6-fluoro-7-{4-[2-hydroxy-2-(3-methoxy-quinolin-5-yl)-ethyl]-piperazin-1-yl}-4-oxo-1,4-dihydro-quinoline-3-carboxylic acid

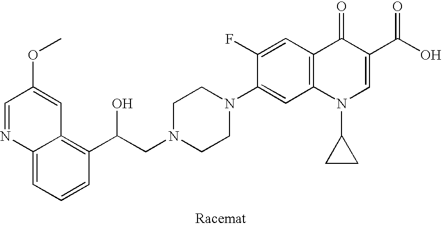

The epoxide (14a) (50 mg) and ciprofloxacine (91.4 mg) were suspended in DMF (0.2 ml) and potassium carbonate (34.3 mg) was added thereto. The reaction mixture was stirred overnight at 100° C. After cooling to room temperature, the reaction mixture was concentrated. The residue was purified by flash chromatography (silica gel, dichloromethane:methanol: 9:1). The concentrated fractions were recrystallized from ether:dichloromethane.

MS (EI): m/z: 533 [M+H]⁺

### Example 72

### 1-Cyclopropyl-6-fluoro-7-(4-{1-[2-hydroxy-2-(3-methoxy-quinolin-5-yl)-ethyl]-piperidin-4-yl}-piperazin-1-yl)-4-oxo-1,4-dihydro-quinoline-3-carboxylic acid (racemate)

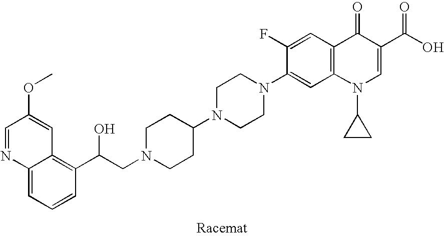

### 72a) 4-(4-Benzyl-piperazin-1-yl)-piperidine-1-carboxylic acid tert-butyl ester

N-tert-Boc 4-piperidinone (5.0 g) and 1-benzyl piperazine (4.43 g) were dissolved in methanol (60 ml) and mixed with acetic acid (1.58 g). The mixture was stirred for 7 hours at room temperature. Subsequently, sodium cyanoborohydride (1.89 g) and methanol (20 ml) were added. The mixture was stirred overnight at room temperature. After the addition of water (250 ml), the mixture was extracted with acetic acid ethyl ester (4×250 ml). The combined organic phases were dried over sodium sulfate, filtered and concentrated. The residue was purified by MPLC and resulted in the desired product (4.52 g).

MS (EI): m/z: 360 [M+H]⁺

### 72b) 4-piperazin-1-yl-piperidine-1-carboxylic acid tert-butyl ester

To a solution of the protected piperazine (72a) (2.05 g) in methanol (20 ml), 10% palladium hydroxide (0.5 g) was added and stirred at room temperature for 48 hours under a hydrogen gas atmosphere. The reaction mixture was filtered, the filtrate was concentrated and the residue was purified by flash chromatography (silica gel; dichloromethane:methanol:ammonia: 19:0.9:0.1, then 9:0.9:0.1, then 4:0.9:0.1, then 3:1.8:0.2) and resulted in the desired product (1.0 mg).

MS (EI): m/z: 270 [M+H]⁺

### 72c) 4-{4-[2-Hydroxy-2-(3-methoxy-quinolin-5-yl)-ethyl]-piperazin-1-yl}-piperidine-1-carboxylic acid tert-butyl ester

The epoxide (14b) (0.39 g), piperazine (72b) (0.29 g), potassium carbonate (0.29 g) and lithium perchlorate (0.12 g) were dissolved in dry DMF (4 ml) and stirred under a nitrogen gas atmosphere for 24 hours at 100° C. The mixture was cooled to room temperature and concentrated. The residue was purified by flash chromatography (silica gel, first of all, dichloromethane, then dichloromethane:methanol:ammonia: 19:0.9:0.1, then 9:0.9:0.1) and resulted in the desired product (472 mg).

MS (EI): m/z: 471 [M+H]⁺

### 72d) 1-(3-Methoxy-quinolin-5-yl)-2-(4-piperidin-4-yl-piperazin-1-yl)-ethanol

To an ice cold solution of the Boc-piperidine (72c) (317 mg) in dichloromethane (10 ml), TFA (1.5 ml) was added in drops. The mixture was stirred for 15 minutes at is 0° C. and then thawed up to room temperature within 45 minutes. The mixture was concentrated, adjusted to an alkaline pH value with ammonia and subsequently extracted with dichloromethane:methanol (9:1) (5×25 ml). The combined organic phases were concentrated and resulted in the desired product (93 mg).

MS (EI): m/z: 371 [M+H]⁺

### 72e) Title Compound

The amine (72d) (80 mg) and 7-chloro-1-cyclopropyl-6-fluoro-1,4-dihydro-4-oxo-3-quinoline carboxylic acid boron diacetate complex (101 mg) were dissolved in NMP (5 ml), and N-ethyl diisopropylamine (0.3 ml) was added in drops. The mixture was stirred for 24 hours at 80° C. After cooling to room temperature, the reaction mixture was concentrated. The residue was stirred for 30 minutes at 0° C. in a 4 N solution of hydrochloric acid in methanol (10 ml), and subsequently, once more for 1 hour at room temperature. The reaction mixture was mixed with acetic acid ethyl ester (50 ml). The precipitate was filtered, washed and dried. 50 mg of the desired product were obtained.

MS (EI): m/z: 616 [M+H]⁺

The maximum inhibiting concentration (maximale Hemm-Konzentration; MHK) (μg/ml) of the substances according to the examples against different organisms was determined: A. baumannii ATCC 19606, E. cloacae ATCC 23355, E. coli ATCC 25922, K. pneumoniae ATCC 27736, P. mirabilis ATCC 29906, P. aeruginosa ATCC 27853, S. maltophilia ATCC 13637, S. aureus ATCC 43300, S. epidermidis ATCC 14990, S. haemolyticus ATCC 29970, E. faecalis ATCC 29212 and E. faecium ATCC 19434.

The substances according to the examples 1-12, 14-15, 17-20, 22-26, 28-53 and 56-71 have a maximum inhibiting concentration (MHK) of less than or equal to 4 μg/ml against at least two of the organisms mentioned above.

1. Compounds of the formula (I):
wherein 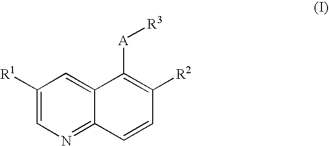
the rest R¹ is a hydrogen atom, a halogen atom, a hydroxy, an amino, a cyano, a nitro, a thiol, a C₁-C₆ alkyl, a C₂-C₆ alkenyl, C₂-C₆ alkynyl, a heteroalkyl, a alkyloxy, a heteroalkyloxy, a cycloalkyloxy, an alkylcycloalkyloxy, a heterocycloalkyloxy or a heteroalkylcycloalkyloxy group;
the rest R² is a hydrogen atom, a halogen atom, a hydroxy, an amino, a cyano, a C₁-C₆ alkyl, a C₂-C₆ alkenyl, C₂-C₆ alkynyl or a heteroalkyl group;
the rest R³ is a group of the following formula: 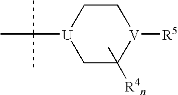
wherein U and V independently of each other are nitrogen atoms or groups of the formula CH or CR⁶;
the rests R⁴ independently of each other are a halogen atom, a hydroxy, an amino, a cyano, a nitro or a thiol group, an alkyl, alkenyl, alkynyl or a heteroalkyl group;
n is equal to 0, 1 or 2;
the rest R⁵ is an alkyl, alkenyl, alkynyl, heteroalkyl, aryl, heteroaryl, cycloalkyl, alkylcycloalkyl, heteroalkylcycloalkyl, heterocycloalkyl, aralkyl or a heteroaralkyl group;
the rests R⁶ independently of each other are a halogen atom, a hydroxy, alkyl, alkenyl, alkynyl or a heteroalkyl group;
A is selected from the following groups: —CR¹⁰(OR¹¹)CR¹²R¹³—, —CR⁸R⁹CR¹⁰(OR¹¹)—, —OCR⁸R⁹CR¹²R¹³—, —CR⁸R⁹CR¹²R¹³O—, —CR⁸R⁹SO₂—, —SO₂CR⁸R⁹—, —CR⁸R⁹NR⁷—, —NR⁷CR⁸R⁹—, —CR⁸R⁹O—, —OCR⁸R⁹—, —SCR⁸R⁹—, —NR⁷C(═O)—, —C(═O)NR⁷— and —CR⁸R⁹CR¹²R¹³—;
the rest R⁷ is a hydrogen atom, a trifluoromethyl, C₁-C₆ alkyl, C₂-C₆ alkenyl, C₁-C₆ alkoxycarbonyl, C₁-C₆ alkylcarbonyl or an carbonylamino group, wherein the amino group of the carbonylamino group, if applicable, may be substituted by a C₁-C₆ alkoxycarbonyl, a C₁-C₆ alkylcarbonyl, a C₂-C₆ alkenyloxycarbonyl, a C₂-C₆ alkenylcarbonyl, a C₁-C₆ alkyl, a C₂-C₆ alkenyl and, if applicable, further may be substituted by a C₁-C₆ alkyl or a C₂-C₆ alkenyl group;
the rests R⁸, R⁹, R¹⁰, R¹² and R¹³ independently of each other are a hydrogen atom, a halogen atom, an azide, a trifluoromethyl, a hydroxy, an amino, a C₁-C₆ alkyloxy, C₁-C₆ alkylthio, C₁-C₆ alkyl, C₂-C₆ alkenyl, C₁-C₆ alkoxycarbonyl, C₂-C₆ alkenyloxycarbonyl, C₁-C₆ alkylsulfonyl, C₂-C₆ alkenylsulfonyl or a sulfonylamino group, wherein the amino group of the sulfonylamino group, if applicable, may be substituted by an C₁-C₆ alkyl or a phenyl group;
the rest R¹¹ is a hydrogen atom, a trifluoromethyl, a C₁-C₆ alkyl, a C₂-C₆ alkenyl, C₁-C₆ alkoxycarbonyl, C₁-C₆ alkylcarbonyl or a carbonylamino group, wherein the amino group of the carbonylamino group, if applicable, may be substituted by a C₁-C₆ alkoxycarbonyl, C₁-C₆ alkylcarbonyl, C₂-C₆ alkenyloxycarbonyl, C₂-C₆ alkenylcarbonyl, C₁-C₆ alkyl, C₂-C₆ alkenyl and, if applicable, further may be substituted by a C₁-C₆ alkyl or a C₂-C₆ alkenyl group;
or a pharmacologically acceptable salt, solvate, hydrate or a pharmacologically acceptable formulation thereof.

2. Compounds according to claim 1, wherein A is selected from the following groups: —CH(OH)CH₂—, —CH₂CH(OH)—, —OCH₂CH₂—, —CH₂CH₂O—, —CH₂SO₂—, —SO₂CH₂—, —CH₂N(C₁-C₄ alkyl)-, —N(C₁-C₄ alkyl)CH₂—, —CH₂NH—, —NHCH₂—, —CH₂O—, —OCH₂—, —CH₂S—, —SCH₂—, —N(C₁-C₄ alkyl)C(═O)—, —C(═O)N(C₁-C₄ alkyl)-, —NHC(═O)—, —C(═O)NH— or —CH₂CH₂—.

3. Compounds according to claim 1 or 2, wherein A is a group of the formula —CH(OH)CH₂— or —OCH₂CH₂—.

4. Compounds according to claim 1, 2 or 3, wherein the rest R¹ is a methoxy group.

5. Compounds according to claim 1, 2, 3 or 4, wherein the rest R² is a hydrogen atom or a halogen atom.

6. Compounds according to claim 1, 2, 3, 4 or 5, wherein the rest R⁵ is a group of the formula —B—Y, wherein B is a bond, an alkylene, an alkenylene, an alkynylene, an —NH—, —NHSO₂—, —SO₂—, —C(═O)—, a heteroalkylene or a heterocycloalkylene group, and Y is an aryl, heteroaryl, aralkyl, heteroaralkyl, cycloalkyl, heterocycloalkyl, alkylcycloalkyl, heteroalkylcycloalkyl, heteroarylheterocycloalkyl or a arylheterocycloalkyl group.

7. Compounds according to claim 6, wherein B is a group of the formula —NH—, —NHCH₂—, —CH₂NH—, —NHCH₂CH₂—, —CH₂CH₂NH—, —NHCH₂CH₂CH₂—, —CH₂CH₂CH₂NH—, —CH₂—, —CH₂CH₂—, —CH₂CH₂CH₂—, —NHC(═O)—, —C(═O)NH—, —CH(OH)—, —CH₂CH(OH)—, —CH(OH)CH₂—, —NHSO₂—, —SO₂NH—, —SO₂—, —C(═S)NH—, —NHC(═S)—, —C(═NOH)—, —CH₂C(═NOH)—, —C(═NOH)CH₂—, —C(═O)—, —C(═O)—C(═O)—, —CH₂C(═O)—, —C(═O)CH₂—, —N(C₁-C₄ alkyl)CH₂—, —CH₂N(C₁-C₄ alkyl)- or a piperazine group.

8. Compounds according to claim 6 or 7, wherein Y has one of the following structures:

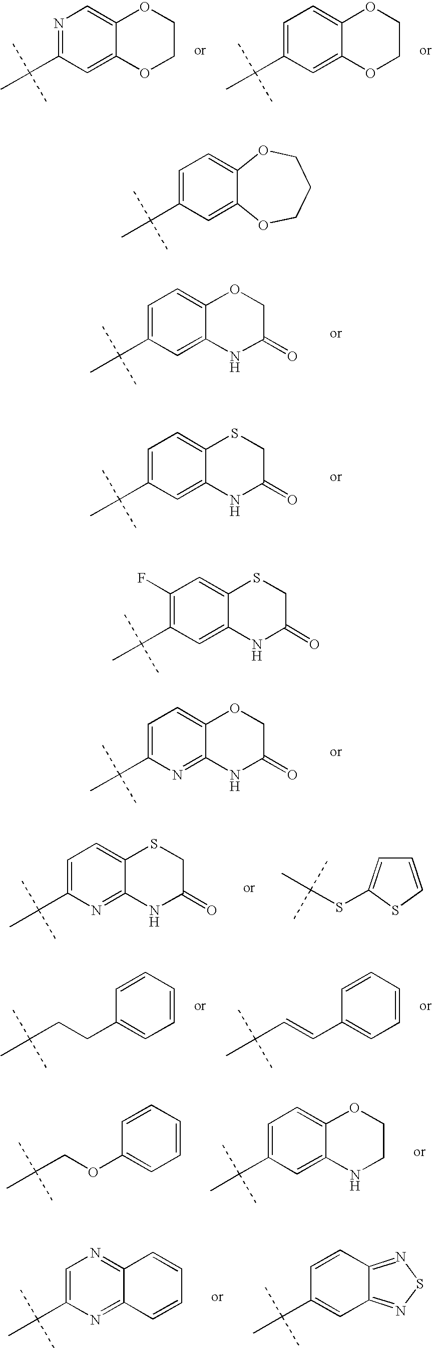

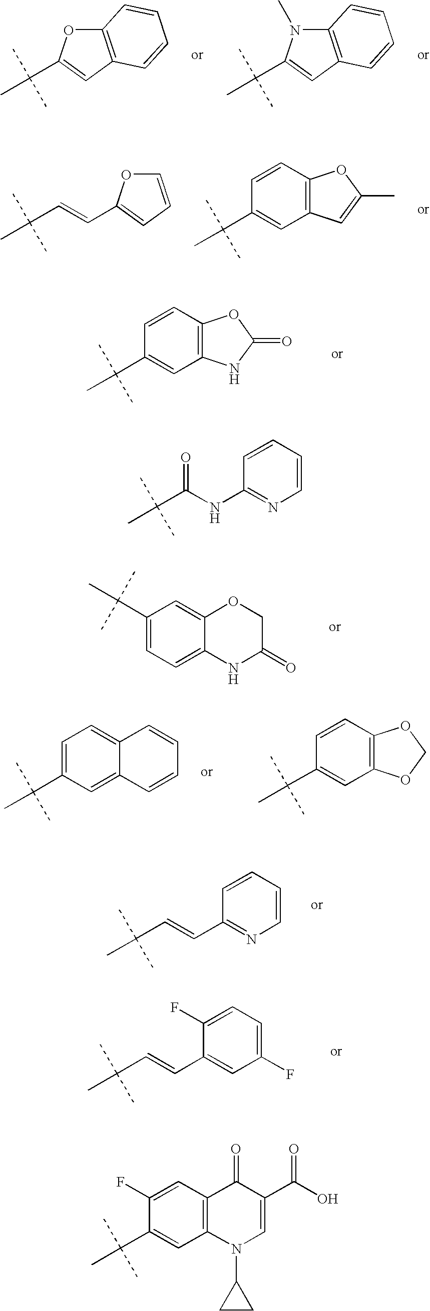

9. Compounds according to claim 1, 2, 3, 4, 5, 6, 7 or 8, wherein the rest R³ is selected from the following groups:

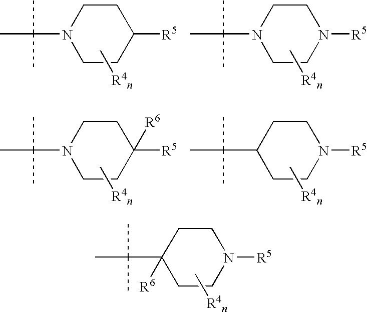

10. Compounds according to claim 9, wherein the rests R⁴ independently of each other are a halogen atom, a hydroxy, a cyano, a C₁-C₄ alkyl or a C₁-C₄ heteroalkyl group.

11. Compounds according to claim 9, wherein the rests R⁶ independently of each other are a halogen atom, a hydroxy, a C₁-C₄ alkyl or a C₁-C₄ heteroalkyl group.

12. Pharmaceutical compositions, which contain a compound according to claims 1 to 11 as an active ingredient, and optionally, carriers and/or adjuvants.

13. Use of a compound or a pharmaceutical composition according to claims 1 to 12 for the treatment of bacterial infections.
